(19) United States
(12) Patent Application Publication
(10) Pub. No.: US 2024/0082403 A1
(43) Pub. Date: Mar. 14, 2024

(54) Chimeric Antigen Receptors against Human Cytomegalovirus

(71) Applicants: The Regents of the University of California, Oakland, CA (US); City of Hope, Duarte, CA (US)

(72) Inventors: Otto O. Yang, Ph.D., Los Angeles, CA (US); Ayub Ali, Porter Ranch, CA (US); Don J. Diamond, Glendora, CA (US); Flavia Chiuppesi, Monrovia, CA (US); Felix Wussow, Monrovia, CA (US)

(21) Appl. No.: 17/766,989

(22) PCT Filed: Oct. 12, 2020
(86) PCT No.: PCT/US2020/055194

§ 371 Date: Apr. 6, 2022

Related U.S. Application Data
(60) Provisional application No. 62/914,408, filed on Oct. 11, 2019.

(51) Int. Cl. A61K 39/00; A61P 31/22; C07K 14/725; C07K 16/08

(52) U.S. Cl. CPC A61K 39/464838; A61K 39/4631; A61P 31/22; C07K 14/7051; C07K 16/089; C07K 2317/622

(57) ABSTRACT

Disclosed herein are CMV-specific CARs. In some embodiments. the present invention is directed to a method of treating, reducing, or inhibiting an infection by a cytomegalovirus in a subject, which comprises administering to the subject (a) an expression vector that encodes a CMV-specific CAR as described herein, or (b) one or more cells that are transduced with the expression vector.

### CROSS-REFERENCE TO RELATED APPLICATIONS

This application claims the benefit of U.S. Patent Application No. 62/914,408, filed Oct. 11, 2019, which is herein incorporated by reference in its entirety.

### ACKNOWLEDGEMENT OF GOVERNMENT SUPPORT

This invention was made with Government support under Grant Number AI103960, awarded by the National Institutes of Health. The Government has certain rights in the invention.

### REFERENCE TO A SEQUENCE LISTING SUBMITTED VIA EFS-WEB

The content of the ASCII text file of the sequence listing named “20201012_034044_209WO1_ST25” which is 85.9 kb in size was created on Oct. 7, 2020 and electronically submitted via EFS-Web herewith the application is incorporated herein by reference in its entirety.

### BACKGROUND OF THE INVENTION

### 1. Field of the Invention Compositions and methods for treating cytomegalovirus infections.

### 2. Description of the Related Art

Human cytomegalovirus (CMV) infection is widespread, ranging from about 60% of adults in the US to nearly 100% in other parts of the world. In most healthy persons, infection is lifelong but immunologically contained and asymptomatic. Some perinatally infected persons, and persons who are significantly immunosuppressed (due to AIDS or iatrogenic/therapeutic immunosuppression for conditions such as bone marrow or organ transplantation) can develop disseminated infection with significant morbidity and mortality due to end organ damage.

The major arm of immunity controlling CMV infection in healthy hosts is cellular immunity, particularly CD8+ T lymphocytes (C8TLs). Autologous immunotherapy using expanded CMV-specific C8TLs has provided proof-of-concept that C8TLs can treat CMV in immunocompromised hosts, but this approach is not generally applicable due to various technical limitations. On the other hand, gene therapy with a chimeric antigen receptor (CAR) targeted against CMV could be readily applied to generate CMV-specific C8TLs in patients, analogous to the growing use of CAR T-cell gene therapy for various cancers.

To date only one CMV-specific CAR has been reported in the literature, and it has not been advanced to clinical trials to our knowledge.

### SUMMARY OF THE INVENTION

“CMV-specific CARs”: In some embodiments, the present invention is directed to a cytomegalovirus specific chimeric antigen receptor (CMV-specific CAR), which comprises a single chain antibody sequence or fragment thereof having SEQ ID NO: 21, SEQ ID NO: 22, SEQ ID NO: 23, SEQ ID NO: 24, SEQ ID NO: 25, and SEQ ID NO: 26 as follows: G-X1-X2-X3-X4-X5-X6-X7-X8-X9 Formula H1 (SEQ ID NO: 21), wherein, X1 is F or Y, X2 is S or T, X3 is L or F, X4 is S or T, X5 is D, I, N, S, or T, X6 is F, Y, or S, X7 is G, Y, or W, X8 is present or absent and if present X8 is L or I, and, X9 is present or absent and if present X9 is G; I-X10-X11-X12-X13-X14-X15-X16 Formula H2 (SEQ ID NO: 22), wherein, X10 is D, N, S, or W, X11 is D, N, P, T, or W, X12 is D, G, N, Y, or S, X13 is D, G, or T, X14 is D, G, N, or S, X15 is E, K, S, or Y, and, X16 is present or absent and if present X16 is P or T; X17-X18-X19-X20-X21-X22-X23-X24-X25-X26-X27-X28-X29-X30-X31 Formula H3 (SEQ ID NO: 23), wherein, X17 is A, S, or V, X18 is R, N, or S, X19 is E, G, K, P, R, or S, X20 is G, H, K, L, Y, or W, X21 is D, L, R, S, or Y, X22 is D, F, G, L, S, or Y, X23 is A, D, F, G, L, P, or Y, X24 is A, D, I, Q, S, V, or Y, X25 is present or absent and if present X25 is E, F, N, S, or Y, X26 is present or absent and if present X26 is A, G, M, L, or P is, X27 present or absent and if present X27 is D, L, or Y, X28 is present or absent and if present X28 is A, F, L, or Y, X29 is present or absent and if present X29 is D, F, G, or M, X30 is present or absent and if present X30 is C, D, or Y, and, X31 is present or absent and if present X31 is S or Y; X32-X33-X34-X35-X36-X37-X38-X39-X40-X41-X42 Formula L1 (SEQ ID NO: 24), wherein, X32 is E, K, or Q, X33 is G or S, X34 is I, L, or V, X35 is D, G, S, or V, X36 is D, H, N, S, or T, X37 is D, N, S, or Y, X38 is present or absent and if present X38 is D, G, or N, X39 is present or absent and if present X39 is G, N, or Y, X40 is present or absent and if present X40 is K, N, or S, X41 is present or absent and if present X41 is F, Y, or T, and, X42 is present or absent and if present X42 is Y; X43-X44-S Formula L2 (SEQ ID NO: 25), wherein, X43 is D, L, R, T, or Y, and, X44 is A, T, or V; X45-X46-X47-X48-X49-X50-P-X51-T Formula L3 (SEQ ID NO: 26), wherein, X45 is S, Q, or W, X46 is H, N, or Q, X47 is D, G, S, or Y, X48 is H, N, R, S, T, or Y, X49 is E, H, K, R, S, or T, X50 is D, F, L, S, V, or W, and, X51 is L, P, W, or Y. In some embodiments, a) Formula H1 (SEQ ID NO: 21) is GFSLSTYGIG (SEQ ID NO: 27), GFSLTTSGLG (SEQ ID NO: 28), GFTFSDYY (SEQ ID NO: 29), GYTFTIYG (SEQ ID NO: 30), GYTFTNFG (SEQ ID NO: 31), GYTFTSYG (SEQ ID NO: 32), GYTFTSYW (SEQ ID NO: 33), GYTFTIYW (SEQ ID NO: 34), or GYTFTSYW (SEQ ID NO: 35); b) Formula H2 (SEQ ID NO: 22) is IDPSDSET (SEQ ID NO: 36), IDPSDSET (SEQ ID NO: 37), IDPSDSET (SEQ ID NO: 38), INTYTGEP (SEQ ID NO: 39), ISDDGNYT (SEQ ID NO: 40), ISNGGGST (SEQ ID NO: 41), IWWDDDK (SEQ ID NO: 42), or IWWNDNK (SEQ ID NO: 43); c) Formula H3 (SEQ ID NO: 23) is AREHYYGINPLLGC (SEQ ID NO: 44), ARGWLLPVFAY (SEQ ID NO: 45), ARKGYYGSSGYFDY (SEQ ID NO: 46), ARRGDGLYSMDY (SEQ ID NO: 47), ARTGYFDV (SEQ ID NO: 48), SNGYSSFAY (SEQ ID NO: 49), VRPKRDFQYLYAMDY (SEQ ID NO: 50), VRSLYDYDEGYYFDS (SEQ ID NO: 51), or ASSGTGAY (SEQ ID NO: 52); d) Formula L1 (SEQ ID NO: 24) is ESIDSYGNSF (SEQ ID NO: 53), KSVSTSGYSY (SEQ ID NO: 54), QGISNY (SEQ ID NO: 55), QSIGNN (SEQ ID NO: 56), QSISDY (SEQ ID NO: 57), QSLVHSNGNTY (SEQ ID NO: 58), QSVSND (SEQ ID NO: 59), QSISNN (SEQ ID NO: 60), or QSLLDSDGKTY (SEQ ID NO: 61); e) Formula L2 (SEQ ID NO: 25) is DTS (SEQ ID NO: 62), LAS (SEQ ID NO: 63), RAS (SEQ ID NO: 64), TVS (SEQ ID NO: 65), YAS (SEQ ID NO: 66), YTS (SEQ ID NO: 67), or LVS (SEQ ID NO: 68); and/or f) Formula L3 (SEQ ID NO: 26) is QHSRELPWT (SEQ ID NO: 69), QNGHTFPPT (SEQ ID NO: 70), QQDYSSPWT (SEQ ID NO: 71), QQSNEDPLT (SEQ ID NO: 72), QQSNRWPWT (SEQ ID NO: 73), QQYSKLPYT (SEQ ID NO: 74), SQSTHVPYT (SEQ ID NO: 75), QQSNSWPLT (SEQ ID NO: 76), or WQGTHFPYT (SEQ ID NO: 77). In some embodiments, Formula H2 (SEQ ID NO: 22) is INTYTGEP (SEQ ID NO: 39) and Formula L2 (SEQ ID NO: 25) is YAS (SEQ ID NO: 66). In some embodiments, the single chain antibody or fragment thereof comprises SEQ ID NO: 30 with 0, 1, 2, or 3 amino acid substitutions, additions, or deletions, SEQ ID NO: 39 with 0, 1, 2, or 3 amino acid substitutions, additions, or deletions, SEQ ID NO: 46 with 0, 1, 2, or 3 amino acid substitutions, additions, or deletions, SEQ ID NO: 59 with 0, 1, 2, or 3 amino acid substitutions, additions, or deletions, SEQ ID NO: 66 with 0, 1, or 2 amino acid substitutions, additions, or deletions, and SEQ ID NO: 71 with 0, 1, 2, or 3 amino acid substitutions, additions, or deletions. In some embodiments, the single chain antibody or fragment thereof comprises SEQ ID NO: 30, SEQ ID NO: 39, SEQ ID NO: 46, SEQ ID NO: 59, SEQ ID NO: 66, and SEQ ID NO: 71. In some embodiments, the single chain antibody or fragment thereof comprises or consists of a VH chain and a VL chain selected from those set forth in Table 1. In some embodiments, the single chain antibody or fragment thereof comprises or consists of SEQ ID NO: 11 and SEQ ID NO: 12. In some embodiments, the CMV-specific CAR comprises or consists of SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91, preferably SEQ ID NO: 89.

In some embodiments, the present invention is directed to a nucleic acid molecule that encodes a CMV-specific CAR as described herein, such as those described in the section entitled “CMV-specific CARs” above.

In some embodiments, the present invention is directed to an expression vector comprising a nucleic acid molecule that encodes a CMV-specific CAR as described herein, such as those described in the section entitled “CMV-specific CARs” above.

In some embodiments, the present invention is directed to a host cell or a progeny cell thereof comprising one or more expression vectors as described herein. In some embodiments, the host cell or the progeny cell is a CD8+ T lymphocyte, hematopoietic stem cell, or a hematopoietic progenitor cell. In some embodiments, the host cell or the progeny cell expresses one or more chimeric antigen receptors encoded by the one or more expression vectors.

In some embodiments, the present invention is directed to a method of treating, reducing, or inhibiting an infection by a cytomegalovirus in a subject, which comprises administering to the subject (a) an expression vector that encodes a CMV-specific CAR as described herein, such as those described in the section entitled “CMV-specific CARs” above, or (b) one or more cells that are transduced with the expression vector.

In some embodiments, the present invention is directed to a method of treating, reducing, or inhibiting an infection by a cytomegalovirus in a subject, which comprises administering to the subject (a) an expression vector that encodes a CMV-specific CAR as described herein, such as those described in the section entitled “CMV-specific CARs” above, or (b) one or more cells that are transduced with the expression vector.

In some embodiments, the present invention is directed to a method of treating, reducing, or inhibiting an infection by a cytomegalovirus in a subject, which comprises transplanting one or more cells that express one or more CMV-specific CARs as described herein, such as those described in the section entitled “CMV-specific CARs” above to the subject.

In some embodiments, the present invention is directed to a method of treating, reducing, or inhibiting an infection by a cytomegalovirus in a subject, which comprises transplanting one or more host cells or progeny cells thereof as described herein in the subject.

In some embodiments, the present invention is directed to a method of killing cells infected with a cytomegalovirus, which comprises contacting the infected cells with one or more cells (a) that express one or more CMV-specific CARs as described herein, such as those described in the section entitled “CMV-specific CARs” above, or (b) comprise an expression vector that encodes the one or more CMV-specific CARs.

In some embodiments, the present invention is directed to a method of reducing replication of a cytomegalovirus in a cell or a subject, which comprises contacting the cell with or administering to the subject one or more cells (a) that express one or more CMV-specific CARs as described herein, such as those described in the section entitled “CMV-specific CARs” above, or (b) comprise an expression vector that encodes the one or more CMV-specific CARs.

In some embodiments, the present invention is directed to (a) the use of one or more CMV-specific CARs as described herein, such as those described in the section entitled “CMV-specific CARs” above, (b) the use of one or more nucleic acid molecules that encode a CMV-specific CAR as described herein, such as those described in the section entitled “CMV-specific CARs” above, (c) the use of one or more expression vectors which comprise a nucleic acid molecule that encodes a CMV-specific CAR as described herein, such as those described in the section entitled “CMV-specific CARs” above, and/or (d) the use of one or more host cells or progeny thereof which comprises one or more expression vectors as described herein.

In some embodiments, the present invention is directed to (a) the use of one or more CMV-specific CARs as described herein, such as those described in the section entitled “CMV-specific CARs” above in the manufacture of a medicament for the treatment of a cytomegalovirus infection, (b) the use of one or more nucleic acid molecules that encode a CMV-specific CAR as described herein, such as those described in the section entitled “CMV-specific CARs” above in the manufacture of a medicament for the treatment of a cytomegalovirus infection, (c) the use of one or more expression vectors which comprise a nucleic acid molecule that encodes a CMV-specific CAR as described herein, such as those described in the section entitled “CMV-specific CARs” above in the manufacture of a medicament for the treatment of a cytomegalovirus infection, and/or (d) the use of one or more host cells or progeny thereof which comprises one or more expression vectors as described herein in the manufacture of a medicament for the treatment of a cytomegalovirus infection.

In some embodiments, the present invention is directed to (a) the use of one or more CMV-specific CARs as described herein, such as those described in the section entitled “CMV-specific CARs” above for the treatment of a cytomegalovirus infection, (b) the use of one or more nucleic acid molecules that encode a CMV-specific CAR as described herein, such as those described in the section entitled “CMV-specific CARs” above for the treatment of a cytomegalovirus infection, (c) the use of one or more expression vectors which comprise a nucleic acid molecule that encodes a CMV-specific CAR as described herein, such as those described in the section entitled “CMV-specific CARs” above for the treatment of a cytomegalovirus infection, and/or (d) the use of one or more host cells or progeny thereof which comprises one or more expression vectors as described herein for the treatment of a cytomegalovirus infection.

In some embodiments, the present invention is directed to (a) one or more CMV-specific CARs as described herein, such as those described in the section entitled “CMV-specific CARs” above, (b) one or more nucleic acid molecules that encode a CMV-specific CAR as described herein, such as those described in the section entitled “CMV-specific CARs” above, (c) one or more expression vectors which comprise a nucleic acid molecule that encodes a CMV-specific CAR as described herein, such as those described in the section entitled “CMV-specific CARs” above, and/or (d) one or more host cells or progeny thereof which comprises one or more expression vectors as described herein, for the treatment of a cytomegalovirus infection.

In some embodiments, the subject is human. In some embodiments, the subject has an immunodeficiency. In some embodiments the cytomegalovirus is a human cytomegalovirus.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1: NAb recognition of CMV-infected EpC. EpC were infected with CMV TB40/E (5 MOI). Cells were fixed and immune-stained with 1B2 (PC-specific) or 62-11 (gH-specific) NAb at 48 hours (middle curves) and 72 hours (right curves) post infection (PI). Controls were uninfected EpC (left curves).

FIG. 2: Schematic of a typical CAR. A single chain antibody serves as the binding domain (gray, top portion), tethered by an immunoglobulin hinge-CH2-CH3 region spacer (white) to the CD8 transmembrane region (black), linked to a 4-1BB and CD3ζ co-signaling and signaling domains (gray, bottom portion).

FIG. 3: Schematic of an exemplary CAR construct.

FIG. 4: CAR expression in transduced primary C8TLs by Western blot

analysis. Primary C8TLs were transduced with lentiviral vectors delivering genes for the indicated CARs. Lysates were then assessed by Western blot staining for CD3ζ. The closed arrow indicates the approximate size of the CAR molecule containing CD3ζ, and the open arrow indicates the approximate size of native (unmodified) CD3ζ. Note that 21F6 and 54E11 CARs are identical duplicates.

FIG. 5A: CAR expression in transduced primary C8TLs by flow cytometry. Primary C8TLs were transduced with lentiviral vectors delivering genes for the indicated CARs. The cells were then stained for cell surface human antibody expression and analyzed by flow cytometry.

FIG. 5B: CAR expression in transduced primary C8TLs by flow cytometry. Primary C8TLs were transduced with lentiviral vectors delivering genes for the indicated CARs. The cells were then stained for cell surface human antibody expression and analyzed by flow cytometry. Not shown: 62-11.

FIG. 6: Recognition of CMV-infected target cells by CAR-transduced C8TLs. 21E9 CAR-transduced primary C8TLs were exposed to CMV-infected or -uninfected target cells and assessed by intracellular IFN-γ/TNF-α and cell surface CD107a staining by flow cytometry.

FIG. 7: Suppression/clearance of CMV by CAR-transduced primary C8TLs. Acutely CMV-infected cells (with a GFP-expressing strain of CMV) were cocultured with no cells, untransduced primary C8TLs, or C8TLs expressing the CAR were added after 4 days of infection. Imaging was done 3 days later. For better reproducibility in black and white, the color of the original figure was inverted.

FIG. 8 to FIG. 11: Intracellular cytokine and cell surface CD107a expression by CAR-transduced primary C8TLs upon exposure to CMV-infected cells.

FIG. 8: Representative dot plots are shown for production of both intracellular IFN-γ and TNF-α by untransduced (top row) or CAR-transduced (bottom row) C8TLs exposed to uninfected (left column) or acutely CMV TR-infected (right column) ARPE-19 cells.

FIG. 9: Representative histograms are shown for cell surface expression of CD107a on untransduced (top) or CAR-transduced (bottom) C8TLs exposed to uninfected (left histogram) or CMV TR-infected (right histogram) ARPE-19 cells.

FIG. 10: The net percentages of untransduced or CAR-transduced C8TLs producing both intracellular IFN-γ and TNF-α in response to CMV-infected target cells (after subtraction of the response to uninfected target cells) are plotted.

FIG. 11: The net percentages of untransduced or CAR-transduced C8TLs expressing cell surface CD107a after exposure to acutely CMV-infected ARPE-19 cells are plotted. Similar results were seen with CMV TB40/E-infected ARPE-19 cells (not shown). These results are representative of three experiments with three different C8TL donors; the other four CARs demonstrated minimal activity in two other experiments (not shown).

FIG. 12: Proliferation of CAR-transduced primary C8TLs upon exposure to CMV-infected cells. Primary C8TLs transduced with the indicated CAR were labeled with CellTrace Violet dye and co-cultured for 6 days with uninfected (open gray histograms) or CMV TB40/E-infected (filled black histograms) ARPE-19 cells and analyzed by flow cytometry for dye expression after seven days. These results are representative of two independent experiments with two different C8TL donors, each performed in biological duplicates.

FIG. 13: Killing of CMV-infected target cells by CAR-transduced primary C8TLs. Background specific lysis of uninfected cells (<6%, except for 2-80 that had background levels of 20% and 32% at effector to target ratios of 20:1 and 40:1 respectively) was subtracted from specific lysis of CMV TB40/E-infected cells. In three independent experiments with three different C8TL donors, only 21E9 exhibited consistent targeted killing of CMV-infected cells.

FIG. 14: Suppression of CMV replication in cell culture by CAR-transduced primary C8TLs. ARPE-19 cells were acutely infected with CMV TR (GFP-expressing) for four days, and then co-cultured with no cells, untransduced primary C8TLs, or CAR-transduced C8TLs (at a ratio of 10 C8TLs per target cell), followed by imaging eight hours later. Similar results were obtained with CMV strain TB40/E (not shown). These results are representative of three independent experiments with three different C8TL donors. For better reproducibility in black and white, the color of the original figure was inverted.

### DETAILED DESCRIPTION OF THE INVENTION

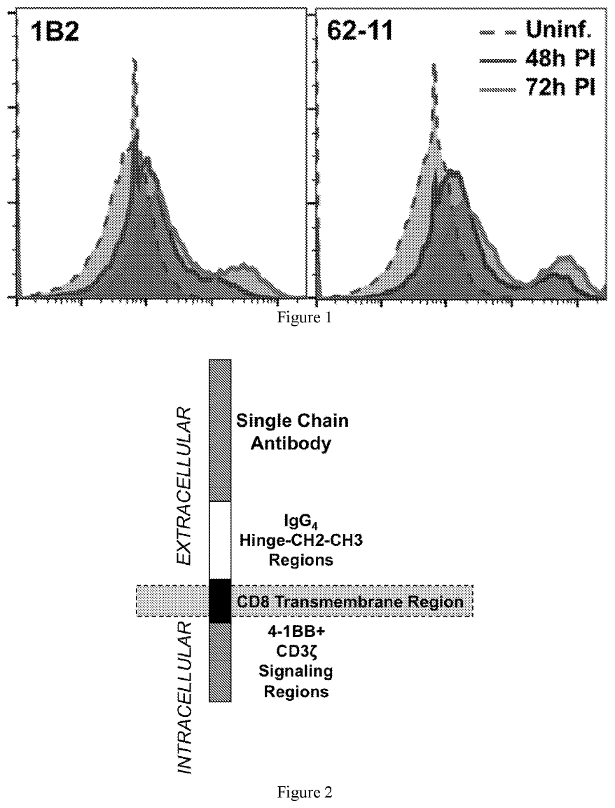

CMV entry into fibroblasts (FB) and epithelial/endothelial cells (EpC/EnC) occurs by alternate routes of entry that are blocked by neutralizing antibodies (NAbs) of various potencies and cell type specificities. CMV infection of FB depends on the major essential envelope glycoprotein complexes (gC) gM/gN, gB, and gH/gL/gO. In contrast to FB entry, CMV infection of EpC/EnC requires an additional complex formed by gH/gL, UL128, UL130, and UL131A (PC). NAb targeting the major gCs block both CMV entry routes; however, NAb recognizing predominantly conformational epitopes formed by two or more of the UL128/UL130/UL131A (UL128/130/131A) subunits of the PC are unable to prevent FB entry, although they are able to interfere with EpC/EnC infection that dramatically exceeds that of NAb targeting the major gC. Both gH and PC-specific antibodies recognize gC expressed on the surface of CMV-infected cells (FIG. 1).

As disclosed herein, engineered cells expressing chimeric antigen receptors (CARs) specific against cytomegalovirus (CMV) exhibit activity against CMV-infected cells and suppress CMV replication. Specifically, primary CD8+ T lymphocytes (primary C8TLs) were transduced with CAR constructs encoding CARs specific against CMV (CMV-specific CARs). As used herein, “C8TLs” include naïve CD8+ T lymphocytes and cytotoxic T cells.

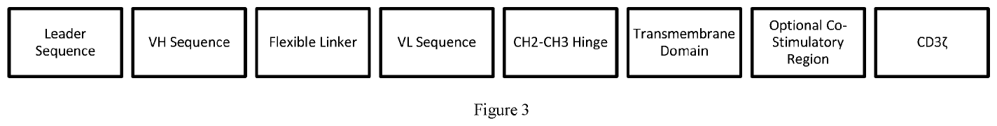

The exemplary CMV-specific CARs comprise a single chain antibody (SCA) or fragment thereof specific against a CMV antigen, a CH2-CH3 hinge region (e.g., from a human IgG4 antibody, a transmembrane domain (e.g., a CD8 transmembrane domain), and a CD3ζ intracellular domain with or without a costimulatory domain (e.g., from 4-1BB or CD28). See FIG. 2.

SCA sequences were derived from the gH and PC-specific NAbs disclosed in US20180230200; Lehmann et al. (2019) J Virol 93(17): e00931-19; Chiuppesi, et al. (2015) J Virol 89(23): 11884-98; and Wussow, et al. (2014) PLOS Pathog 10(11):e1004524, which are herein incorporated by reference in their entirety.

The amino acid sequences of the VH and VL chains (with the CDR sequences underlined) of the antibodies used to construct the SCA sequences of the exemplary CMV-specific CARs are provided in Table 1:

TABLE 1

| MAb Clone | SEQ ID NO: | Sequence |
|---|---|---|
| 1B2-VH | 1 | EVQLVESGGVLVKPGGSLKLSCAASGFTFSDYYMYWVRQTPEKRLE |
| | | WVATISDDGNYTNYPDSVKGRFTISRDNAKNNLYLQMSSLKSEDTA |
| | | MYYCARGWLLPVFAYWGQGTLVTVSA |
| 1B2-VL | 2 | DIVITQSPATLSVTPGDSVSLSCRASQSIGNNLHWYQQKSHESPRL |
| | | LIKYTSQSISGIPSRFSGSGSGTDFTENINSVETEDFGVYFCQQSN |
| | | RWPWTFGGGTKLEIK |
| 54E11-VH | 3 | QIQLVQSGPELKKPGETVKISCKASGYTFTSYGMNWVKQAPGKGLK |
| (21F6-VH) | | WMGWINTYTGEPTYADDFKGRFAFSLETSASTAYLQINNLKNEDTA |
| | | TYFCAREHYYGINPLLGCWGQGTTLTVSS |
| 54E11-VL | 4 | DIQMTQTTSSLSASLGDRVTISCSASQGISNYLNWYQQKPDGTVKL |
| (21F6-VL) | | LIYDTSSLHSGVPSRFSGSGSGTDYSITISNLEPEDIATYYCQQYS |
| | | KIPYTFGGGTKLEIK |
| 12E2-VH | 5 | EVKLVESGGGLVQPGGSLKLSCATSGFTFSDYYMFWVRQTPEKKLE |
| | | WVAYISNGGGSTYYPDTVKGRFTISRDNDKNTLYLQMSRLKSDDTA |
| | | LYYCVRPKRDFQYLYAMDYWGQGTSVTVSS |
| 12E2-VL | 6 | DIVITQSPASLAVSIGQRATISCRASKSVSTSGYSYMHWYQQKPGQ |
| | | SPKLLIYLASNLESGVPARFSGSGSGTDETLNIHPVEDEDAATYYC |
| | | QHSRELPWTFGGGTKLEIK |
| 13B5-VH | 7 | QVTLKESGPGILKPSQTLSLTCSFSGFSLTTSGLGVGWIRQPSGKG |
| | | LEWLAHIWWDDDKYFNPSLRNQLTISKDTSRNQVFLEITSVTTADT |
| | | ATYYCVRSLYDYDEGYYFDSWGQGTTLTVSS |
| 13B5-VL | 8 | EIVMIQSPATLSVNPGDRVSLSCRASQSISDYLHWYQQKSHESPRL |
| | | LIKYASQSISGIPSRFSGSGSGSDFTLSINSVEPEDVGVYYCQNGH |
| | | TFPPTFGGGTKLEIK |
| 18F10-VH | 9 | QVTLKESGPGILQPSQTLSLTCSFSGFSLSTYGIGIGWIRQPSGKG |
| | | LEWLAHIWWNDNKNYNTALKSRLTISKDPSNNQVFLKIASVDTADT |
| | | ATYFCARTGYFDVWGAGTTVTVSS |
| 18F10-VL | 10 | DVVLTQTPLSLPVSLGDQVSISCSSSQSLVHSNGNTYIHWYLQKPG |
| | | QSPKLLIYTVSNRFSGVPDRFSGSGSGTDFTLKISRVEAEDLGLYF |
| | | CSQSTHVPYTFGGGTKLEIK |
| 21E9-VH | 11 | QIQLVQSGPELKKPGETVKISCKASGYTFTIYGMNWVKQAPGKGLK |
| | | WMGWINTYTGEPTYADDFRGRFAFSLETSASTAYLQINNLKNEDTA |
| | | TYFCARKGYYGSSGYFDYWGQGTTLTVSS |
| 21E9-VL | 12 | SIVMTQTPKFLLVSAGDRVTITCKASQSVSNDVSWYQQKPGQSPKL |
| | | LIYYASNRYTGVPDRFTGSGYGTDFTFTISTVQAEDLAVYFCQQDY |
| | | SSPWTFGGGTKLEIK |
| 62-11-VH | 13 | QVQLQQPGAELVRPGASVKLSCKASGYTFTSYWMNWVKQRPGQGLE |
| | | WIGMIDPSDSETHYNQMFKDKATLTVDKSSSTAYMQLSSLTSEDSA |
| | | VYYCSNGYSSFAYWGQGTLVTVSV |
| 62-11-VL | 14 | D(V/I)QMTQTTSSLSASEGDRVTISCSASQGISNYLNWYQQKPDG |
| | | TVKLLIYDTSSLHSGVPSRFSGSGSGTDYSLTISNLEPEDIATYYC |
| | | QQYSKLPYTFGGGTKLEIK |
| 2-80-VH | 15 | QIQLVQSGPELKKPGETVKISCKASGYTFTNFGMNWVKQAPGKGLK |
| | | WMGWINTYTGEPTYADDFKGRFAFSLETSASTASLQINNLKNEDTA |
| | | TYFCARRGDGLYSMDYWGQGTSVTVSS |
| 2-80-VL | 16 | DIVLTQSPASLAVSLGQRATISCRASESIDSYGNSFMYWYQQKPGQ |
| | | PPKLLIYRASNLESGIPARFSGSGSRTDFTLTINPVEADDVATYYC |
| | | QQSNEDPLTFGAGTKLELK |
| 4A3-VH | 17 | QVQLQQPGPELVRPGASVKLSCKASGYTFTIYWMNWVKQRPGQGLE |
| | | WIGMIDPSDSETHYNQMFKDKATLTVDKSSSTAYMQLSSLTSEDSA |
| | | VYYCASSGTGAYWGQGTLLTVSA |
| 4A3-VL | 18 | DVVMTQTPLTLSVTIGQPASISCKSSQSLLDSDGKTYLNWLLQRPG |
| | | QSPKRLIYLVSKLDSGVPDRFTGSGSGTDFTLKISRLEAEDLGVYY |
| | | CWQGTHFPYTFGGGTKLEIK |
| 62-100-VH | 19 | QVQLQQPGAELVRPGASVKLSCKASGYTFTSYWMNWVKQRPGQGLE |
| | | WIGMIDPSDSETHYNQMFKDKATLTVDKSSSTAYMQLSSLTSEDSA |
| | | VYYCSNGYSSFAYWGQGTLVTVSV |
| 62-100-VL | 20 | DIVLTQSPATLSVTPGDSVSLSCRASQSISNNLHWYQQKSHESPRL |
| | | LIKYASQSISGIPSRFSGSGSGTDFTLSINSVETEDFGKYVCQQSN |
| | | SWPLTFGSGTKLEIK |

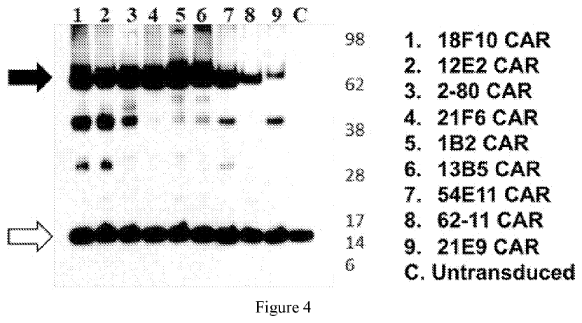

CAR constructs encoding CMV-specific CARs with the SCA sequences were produced. As used herein, a “CAR construct” refers to an expression vector designed to be capable of expression of a given CAR construct, such as a CMV-specific CAR, in a cell when provided therein. The CAR constructs were inserted into lentiviral vectors to deliver these CAR gene sequences to cells. FIG. 3 schematically shows an exemplary CAR construct.

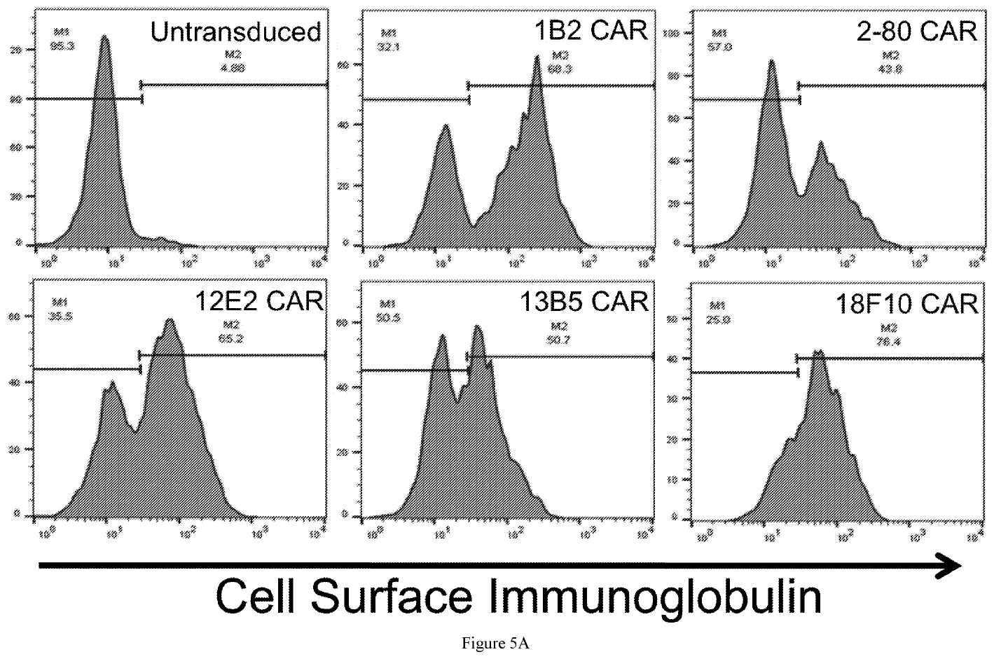

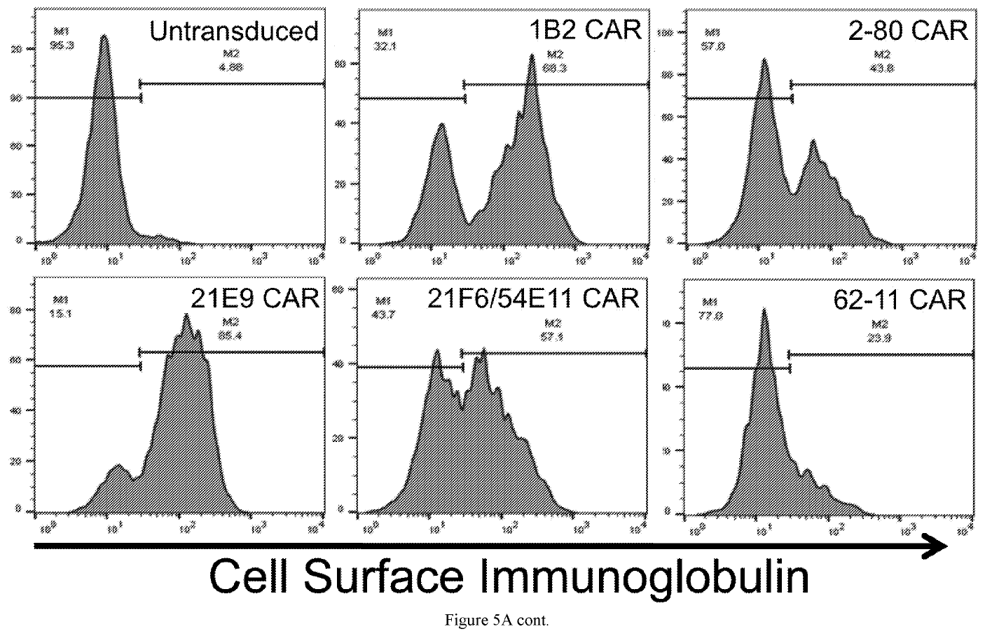

When vectors comprising the CAR constructs were transduced into purified primary C8TLs, expression of the CMV-specific CARs was detected by both Western blot via staining for the CD3ζ component (FIG. 4), and flow cytometry assessing for cell-surface expressed CMV-specific CARs by staining for the SCA sequence, since T cells do not normally have cell surface antibody expression (FIG. 5A & B). These results show that CMV-specific CARs were successfully expressed in primary C8TLs.

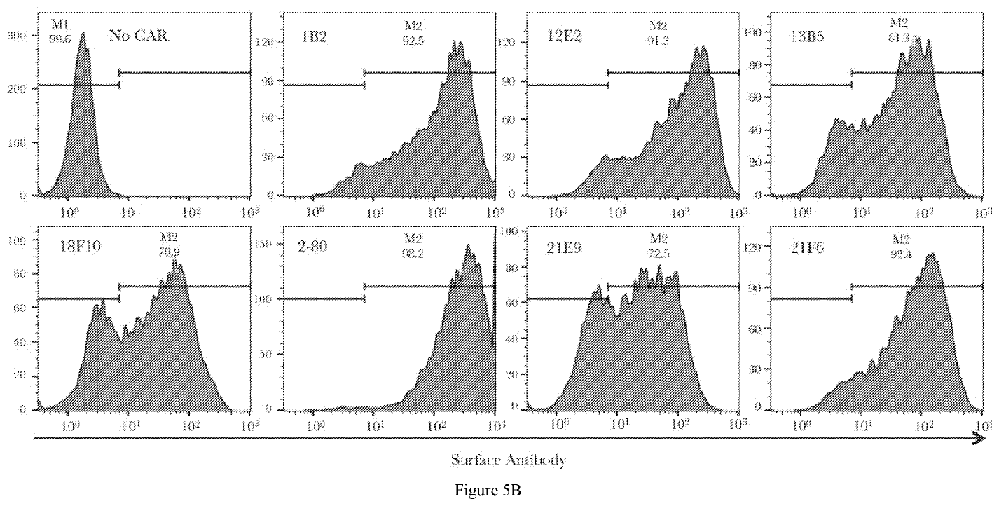

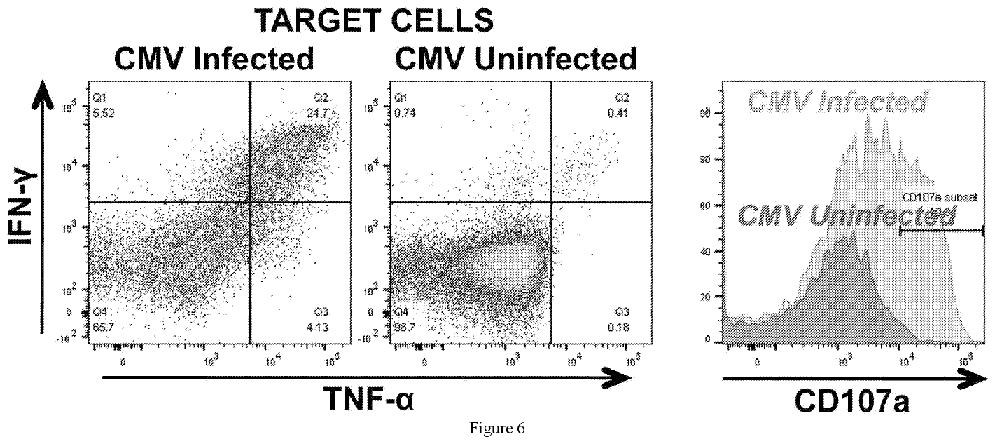

Functional testing of enriched primary C8TLs transduced with CMV-specific CARs confirmed their ability to recognize CMV-infected cells and mediate antiviral activity. When C8TLs transduced with CMV-specific CARs were exposed to CMV-infected cells, the C8TLs released effector cytokines and expressed a marker of cytolytic activity in a CMV-specific manner (FIG. 6), thereby indicating that the CMV-specific CAR functionally directed their recognition of CMV-infected cells. When added to cells infected with a GFP-expressing strain of CMV, the transduced primary C8TLs markedly reduced the concentration of infected cells (FIG. 7), further confirming specific recognition and triggering of antiviral activity by the CMV-specific CARs.

Therefore, in some embodiments, the present invention is directed to a CAR construct encoding a CMV-specific CAR, which comprises a single chain antibody sequence or fragment thereof having SEQ ID NO: 21, SEQ ID NO: 22, SEQ ID NO: 23, SEQ ID NO: 24, SEQ ID NO: 25, and SEQ ID NO: 26 as follows:

Formula H1
(SEQ ID NO: 21)
G-X1-X2-X3-X4-X5-X6-X7-X8-X9

wherein, X1 is F or Y, X2 is S or T, X3 is L or F, X4 is S or T, X5 is D, I, N, S, or T, X6 is F, Y, or S, X7 is G, Y, or W, X8 is present or absent and if present, X8 is L or I, and X9 is present or absent and if present, X9 is G.

Formula H2
(SEQ ID NO: 22)
I-X10-X11-X12-X13-X14-X15-X16

wherein X10 is D, N, S, or W, X11 is D, N, P, T, or W, X12 is D, G, N, Y, or S, X13 is D, G, or T, X14 is D, G, N, or S, X15 is E, K, S, or Y, and X16 is present or absent and if present X16 is P or T.

Formula H3
(SEQ ID NO: 23)
X17-X18-X19-X20-X21-X22-X23-X24-X25-X26-X27-X28-
X29-X30-X31

wherein X17 is A, S, or V, X18 is R, N, or S, X19 is E, G, K, P, R, or S, X20 is G, H, K, L, Y, or W, X21 is D, L, R, S, or Y X22 is D, F, G, L, S, or Y X23 is A, D, F, G, L, P, or Y, X24 is A, D, I, Q, S, V, or Y, X25 is present or absent and if present, X25 is E, F, N, S, or Y, X26 is present or absent and if present, X26 is A, G, M, L, or P is, X27 present or absent and if present, X27 is D, L, or Y, X28 is present or absent and if present, X28 is A, F, L, or Y, X29 is present or absent and if present, X29 is D, F, G, or M, X30 is present or absent and if present, X30 is C, D, or Y, and X31 is present or absent and if present, X31 is S or Y.

Formula L1
(SEQ ID NO: 24)
X32-X33-X34-X35-X36-X37-X38-X39-X40-X41-X42

wherein X32 is E, K, or Q, X33 is G or S X34 is I, L, or V, X35 is D, G, S, or V, X36 is D, H, N, S, or T, X37 is D, N, S, or Y, X38 is present or absent and if present X38 is D, G, or N, X39 is present or absent and if present X39 is G, N, or Y, X40 is present or absent and if present X40 is K, N, or S, X41 is present or absent and if present X41 is F, Y, or T, and X42 is present or absent and if present X42 is Y.

Formula L2
(SEQ ID NO: 25)
X43-X44-S

wherein X43 is D, L, R, T, or Y, and X44 is A, T, or V.

Formula L3
(SEQ ID NO: 26)
X45-X46-X47-X48-X49-X50-P-X51-T

wherein X45 is S, Q, or W, X46 is H, N, or Q, X47 is D, G, S, or Y, X48 is H, N, R, S, T, or Y, X49 is E, H, K, R, S, or T, X50 is D, F, L, S, V, or W, and X51 is L, P, W, or Y.

In some embodiments, SEQ ID NO: 21 is GFSLSTYGIG (SEQ ID NO: 27), GFSLTTSGLG (SEQ ID NO: 28), GFTFSDYY (SEQ ID NO: 29), GYTFTIYG (SEQ ID NO: 30), GYTFTNFG (SEQ ID NO: 31), GYTFTSYG (SEQ ID NO: 32), GYTFTSYW (SEQ ID NO: 33), GYTFTIYW (SEQ ID NO: 34), or GYTFTSYW (SEQ ID NO: 35), preferably SEQ ID NO: 30.

In some embodiments, SEQ ID NO: 22 is IDPSDSET (SEQ ID NO: 36), IDPSDSET (SEQ ID NO: 37), IDPSDSET (SEQ ID NO: 38), INTYTGEP (SEQ ID NO: 39), ISDDGNYT (SEQ ID NO: 40), ISNGGGST (SEQ ID NO: 41), IWWDDDK (SEQ ID NO: 42), or IWWNDNK (SEQ ID NO: 43), preferably SEQ ID NO: 39.

In some embodiments, SEQ ID NO: 23 is AREHYYGINPLLGC (SEQ ID NO: 44), ARGWLLPVFAY (SEQ ID NO: 45), ARKGYYGSSGYFDY (SEQ ID NO: 46), ARRGDGLYSMDY (SEQ ID NO: 47), ARTGYFDV (SEQ ID NO: 48), SNGYSSFAY (SEQ ID NO: 49), VRPKRDFQYLYAMDY (SEQ ID NO: 50), VRSLYDYDEGYYFDS (SEQ ID NO: 51), or ASSGTGAY (SEQ ID NO: 52), preferably SEQ ID NO: 46.

In some embodiments, SEQ ID NO: 24 is ESIDSYGNSF (SEQ ID NO: 53), KSVSTSGYSY (SEQ ID NO: 54), QGISNY (SEQ ID NO: 55), QSIGNN (SEQ ID NO: 56), QSISDY (SEQ ID NO: 57), QSLVHSNGNTY (SEQ ID NO: 58), QSVSND (SEQ ID NO: 59), QSISNN (SEQ ID NO: 60), or QSLLDSDGKTY (SEQ ID NO: 61), preferably SEQ ID NO: 59.

In some embodiments, SEQ ID NO: 25 is DTS (SEQ ID NO: 62), LAS (SEQ ID NO: 63), RAS (SEQ ID NO: 64), TVS (SEQ ID NO: 65), YAS (SEQ ID NO: 66), YTS (SEQ ID NO: 67), or LVS (SEQ ID NO: 68), preferably SEQ ID NO: 66.

In some embodiments, SEQ ID NO: 26 is QHSRELPWT (SEQ ID NO: 69), QNGHTFPPT (SEQ ID NO: 70), QQDYSSPWT (SEQ ID NO: 71), QQSNEDPLT (SEQ ID NO: 72), QQSNRWPWT (SEQ ID NO: 73), QQYSKLPYT (SEQ ID NO: 74), SQSTHVPYT (SEQ ID NO: 75), QQSNSWPLT (SEQ ID NO: 76), or WQGTHFPYT (SEQ ID NO: 77), preferably SEQ ID NO: 67.

In some embodiments, the VH sequence of the SCA comprises SEQ ID NOs: 30, 39, and 46.

In some embodiments, the VL sequence of the SCA comprises SEQ ID NOs: 59, 66, and 71.

In some embodiments, the SCA has a VH sequence comprising SEQ ID NOs: 30, 39, and 46, and a VL sequence comprising SEQ ID NOs: 59, 66, and 71.

In some embodiments, the VH sequence of the SCA is SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, or SEQ ID NO: 19, preferably SEQ ID NO: 11.

In some embodiments, the VL sequence of the SCA is SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, or SEQ ID NO: 20, preferably SEQ ID NO: 12.

In some embodiments, the SCA has a VH sequence comprising SEQ ID NO: 11, and a VL sequence comprising SEQ ID NO: 12.

In some embodiments, the Leader Sequence has 90, 91, 92, 93, 94, 95, 96, 97, 99, or 100% sequence identity to MLLLVTSLLLCELPHPAFLLIP (SEQ ID NO: 78).

In some embodiments, the Flexible Linker has 90, 91, 92, 93, 94, 95, 96, 97, 99, or 100% sequence identity to SGGGGSGGGGSGGGGS (SEQ ID NO: 79).

In some embodiments, the IgG4 hinge-CH2-CH3 region spacer has 90, 91, 92, 93, 94, 95, 96, 97, 99, or 100% sequence identity to

(SEQ ID NO: 80)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

In some embodiments, the CD8 transmembrane region has 90, 91, 92, 93, 94, 95, 96, 97, 99, or 100% sequence identity to DIYIWAPLAGTCGVLLLSL VITLYC (SEQ ID NO: 81).

In some embodiments, the 4-1BB co-signaling region has 90, 91, 92, 93, 94, 95, 96, 97, 99, or 100% sequence identity to

(SEQ ID NO: 82)
KRGRKKLLYIFKQPFMRPVQTTQEEDGCSCRFPEEEEGGCEL.

In some embodiments, the CD3ζ signaling region has 90, 91, 92, 93, 94, 95, 96, 97, 99, or 100% sequence identity to

(SEQ ID NO: 83)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

The sequences of the CMV-specific CARs exemplified herein are:

1B2 CAR:
(SEQ ID NO: 84)
MLLLVTSLLLCELPHPAFLLIPEVOLVESGGVLVKPGGSLKLSCAASGFT
FSDYYMYWVRQTPEKRLEWVATISDDGNYTNYPDSVKGRFTISRDNAKNN
LYLQMSSLKSEDTAMYYCARGWLLPVFAYWGQGTLVTVSASGGGGSGGGG
SGGGGSDIVLTQSPATLSVTPGDSVSLSCRASQSIGNNLHWYQQKSHESP
RLLIKYTSQSISGIPSRFSGSGSGTDFTLNINSVETEDFGVYFCQQSNRW
PWTFGGGTKLEIKESKYGPPCPPCPAPEFLGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGKDIYIWAPL
AGTCGVLLLSLVITLYCKRGRKKLLYIFKQPFMRPVQTTQEEDGCSCRFP
EEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQ
GLSTATKDTYDALHMQALPPR
2-80 CAR:
(SEQ ID NO: 85)
MLLLVTSLLLCELPHPAFLLIPQIQLVQSGPELKKPGETVKISCKASGYT
FTNFGMNWVKQAPGKGLKWMGWINTYTGEPTYADDFKGRFAFSLETSAST
ASLQINNLKNEDTATYFCARRGDGLYSMDYWGQGTSVTVSSSGGGGSGGG
GSGGGGSDIVLTQSPASLAVSLGQRATISCRASESIDSYGNSFMYWYQQK
PGQPPKLLIYRASNLESGIPARFSGSGSRTDFTLTINPVEADDVATYYCQ
QSNEDPLTFGAGTKLELKESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKDIY
IWAPLAGTCGVLLLSLVITLYCKRGRKKLLYIFKQPFMRPVQTTQEEDGC
SCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGH
DGLYQGLSTATKDTYDALHMQALPPR
12E2 CAR:
(SEQ ID NO: 86)
MLLLVTSLLLCELPHPAFLLIPEVKLVESGGGLVQPGGSLKLSCATSGFT
FSDYYMFWVRQTPEKKLEWVAYISNGGGSTYYPDTVKGRFTISRDNDKNT
LYLOMSRLKSDDTALYYCVRPKRDFQYLYAMDYWGQGTSVTVSSSGGGGS
GGGGSGGGGSDIVLTQSPASLAVSLGQRATISCRASKSVSTSGYSYMHWY
QQKPGQSPKLLIYLASNLESGVPARFSGSGSGTDFTLNIHPVEDEDAATY
YCQHSRELPWTFGGGTKLEIKESKYGPPCPPCPAPEFLGGPSVFLFPPKP
KDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPV
LDSDGSFFLYSRLTVDKSRWQEGNVFSCSVMHEALHNHYTQKSLSLSLGK
DIYIWAPLAGTCGVLLLSLVITLYCKRGRKKLLYIFKQPFMRPVQTTQEE
DGCSCRFPEEEEGGCELRVKFSRSADAPAYKQGQNQLYNELNLGRREEYD
VLDKRRGRDPEMGGKPRRKNPQEGLYNELQKDKMAEAYSEIGMKGERRRG
KGHDGLYQGLSTATKDTYDALHMQALPPR
13B5 CAR:
(SEQ ID NO: 87)
MLLLVTSLLLCELPHPAFLLIPQVTLKESGPGILKPSQTLSLTCSFSGFS
LTTSGLGVGWIRQPSGKGLEWLAHIWWDDDKYFNPSLRNQLTISKDTSRN
QVFLEITSVTTADTATYYCVRSLYDYDEGYYFDSWGQGTTLTVSSSGGGG
SGGGGSGGGGSEIVMIQSPATLSVNPGDRVSLSCRASQSISDYLHWYQQK
SHESPRLLIKYASQSISGIPSRFSGSGSGSDFTLSINSVEPEDVGVYYCO
NGHTFPPTFGGGTKLEIKESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKDIY
IWAPLAGTCGVLLLSLVITLYCKRGRKKLLYIFKQPFMRPVQTTQEEDGC
SCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGH
DGLYQGLSTATKDTYDALHMQALPPR
18F10 CAR:
(SEQ ID NO: 88)
MLLLVTSLLLCELPHPAFLLIPQVTLKESGPGILQPSQTLSLTCSFSGFS
LSTYGIGIGWIRQPSGKGLEWLAHIWWNDNKNYNTALKSRLTISKDPSNN
QVFLKIASVDTADTATYFCARTGYFDVWGAGTTVTVSSSGGGGSGGGGSG
GGGSDVVLTQTPLSLPVSLGDQVSISCSSSQSLVHSNGNTYIHWYLQKPG
QSPKLLIYTVSNRFSGVPDRFSGSGSGTDFTLKISRVEAEDLGLYFCSQS
THVPYTFGGGTKLEIKESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKDIYIW
APLAGTCGVLLLSLVITLYCKRGRKKLLYIFKQPFMRPVQTTQEEDGCSC
RFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPR
21E9 CAR:
(SEQ ID NO: 89)
MLLLVTSLLLCELPHPAFLLIPQIQLVQSGPELKKPGETVKISCKASGYT
FTIYGMNWVKQAPGKGLKWMGWINTYTGEPTYADDFRGRFAFSLETSAST
AYLQINNLKNEDTATYFCARKGYYGSSGYFDYWGQGTTLTVSSSGGGGSG
GGGSGGGGSSIVMTQTPKFLLVSAGDRVTITCKASQSVSNDVSWYQQKPG
QSPKLLIYYASNRYTGVPDRFTGSGYGTDFTFTISTVQAEDLAVYFCQQD
YSSPWTFGGGTKLEIKESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKDIYIW
APLAGTCGVLLLSLVITLYCKRGRKKLLYIFKQPFMRPVQTTQEEDGCSC
RFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPR
21F6_54E11 (21F6) CAR:
(SEQ ID NO: 90)
MLLLVTSLLLCELPHPAFLLIPQIQLVQSGPELKKPGETVKISCKASGYT
FTSYGMNWVKQAPGKGLKWMGWINTYTGEPTYADDFKGRFAFSLETSAST
AYLQINNLKNEDTATYFCAREHYYGINPLLGCWGQGTTLTVSSSGGGGSG
GGGSGGGGSDIQMTQTTSSLSASLGDRVTISCSASQGISNYLNWYQQKPD
GTVKLLIYDTSSLHSGVPSRFSGSGSGTDYSLTISNLEPEDIATYYCQQY
SKLPYTFGGGTKLEIKESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGKDIYIW
APLAGTCGVLLLSLVITLYCKRGRKKLLYIFKQPFMRPVQTTQEEDGCSC
RFPEEEEGGCELRVKFSRSADAPAYKQGQNQLYNELNLGRREEYDVLDKR
RGRDPEMGGKPRRKNPQEGLYNELQKDKMAEAYSEIGMKGERRRGKGHDG
LYQGLSTATKDTYDALHMQALPPR
62-11 CAR:
(SEQ ID NO: 91)
MLLLVTSLLLCELPHPAFLLIPQVQLQQPGAELVRPGASVKLSCKASGYT
FTSYWMNWVKQRPGQGLEWIGMIDPSDSETHYNQMFKDKATLTVDKSSST
AYMQLSSLTSEDSAVYYCSNGYSSFAYWGQGTLVTVSSSGGGGSGGGGSG
GGGSDIQMTQTTSSLSASLGDRVTISCSASQGISNYLNWYQQKPDGTVKL
LIYDTSSLHSGVPSRFSGSGSGTDYSLTISNLEPEDIATYYCQQYSKLPY
TFGGGTKLEIKESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGKDIYIWAPLAG
TCGVLLLSLVITLYCKRGRKKLLYIFKQPFMRPVQTTQEEDGCSCRFPEE
EEGGCELRVKFSRSADAPAYKQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR

Therefore, in some embodiments, the CMV-specific CAR comprises SEQ ID NO: 84, SEQ ID NO: 85, SEQ ID NO: 86, SEQ ID NO: 87, SEQ ID NO: 88, SEQ ID NO: 89, SEQ ID NO: 90, or SEQ ID NO: 91, preferably the CMV-specific CAR comprises SEQ ID NO: 89.

In some embodiments, C8TLs transduced with CMV-specific CARs may be administered to subjects. In some embodiments, stem cells, such as hematopoietic stem and progenitor cells (HSPCs), may be transduced with CAR constructs encoding CMV-specific CARs and engrafted in subjects to be treated. As used herein, “HSPC-based CAR cells” refer to a cell engineered to express a CAR by transducing a HSPC with a CAR construct and progeny thereof. As used herein, “HSPC” refers to a hematopoietic stem cell (HSC) and/or a hematopoietic progenitor cell (HPC).

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., (1990) J Mol Biol 215:403-410. Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al., (1997) Nucleic Acids Res 25:3389-3402; and Altschul, et al., (2005) FEBS J 272:5101-5109.

Optimal alignment of sequences for comparison can be conducted, e.g., by the local homology algorithm of Smith & Waterman, Adv Appl Math 2:482 (1981), by the homology alignment algorithm of Needleman & Wunsch, J Mol Biol 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection.

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus.

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab' fragments, F(ab')₂, disulfide linked Fvs (sdFvs), Fvs, and fragments comprising or alternatively consisting of, either a VL or a VH domain.

In some embodiments, the antibodies are monoclonal antibodies. In some embodiments, the monoclonal antibodies are obtained from rabbit-based hybridomas. As used herein, a compound (e.g., receptor or antibody) “specifically binds” a given target (e.g., ligand) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure.

As used herein, “binding affinity” refers to the propensity of a compound to associate with (or alternatively dissociate from) a given target and may be expressed in terms of its dissociation constant, Kd. In some embodiments, the antibodies have a Kd of 10⁻⁵ or less, 10⁻⁶ or less, preferably 10⁻⁷ or less, more preferably 10⁻⁸ or less, even more preferably 10⁻⁹ or less, and most preferably 10⁻¹⁰ or less, to their given target. Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of the antibody for the given target is at least 1-fold or more, preferably at least 5-fold or more, more preferably at least 10-fold or more, and most preferably at least 100-fold or more than its binding affinity for the specified alternative.

The following examples are intended to illustrate but not to limit the invention.

### EXAMPLES

Eight novel CMV-specific CARs were constructed using anti-CMV neutralizing antibody sequences that target the pentameric complex (PC) The CMV-specific CARs were transduced via lentiviral vector into primary C8TLs. Activity against CMV-infected target cells was assessed by release of cytokines (interferon-γ and tumor necrosis factor-α, upregulation of surface CD107a, proliferation, cytolysis of infected cells, and suppression of viral replication. While some CARs showed varying functional activity across these assays, one CMV-specific CAR based on antibody 21E9 was consistently superior in all measures. These results support development of a CMV-specific CAR for therapeutic use against CMV and potentially other applications harnessing CMV-driven immunotherapies.

### Materials and Methods

### Anti-CMV Antibody Sequences

The full variable region sequences of neutralizing antibodies against CMV were utilized to create single chain antibody genes synthesized as codon optimized genes coding for the heavy chain and light chain variable regions (Table 1) separated by a linker, additionally with an upstream leader sequence from granulocyte-macrophage colony-stimulating factor.

### Cell Lines

The cell lines 293T and ARPE-19 were maintained Dulbecco's modified essential medium supplemented with L-glutamine (2 mM), penicillin (100 U/mL), streptomycin (100 U/mL), and heat-inactivated fetal bovine serum (10%) using methods in the art. Primary C8TLs from healthy donors were generated from whole peripheral blood mononuclear cells (PBMCs); in brief they were purified with the MACS Column Separation Kit by positive selection according to the manufacturer's protocol (Miltenyi, San Diego, CA) and stimulated for five days with an anti-CD3 antibody in the presence of irradiated feeder PBMC and 50 U/mL recombinant human interleukin-2 (NIH AIDS Reagent Repository), yielding >95% pure CD3⁺/CD8⁺ cells. Experiments were repeated using PBMCs from three healthy donors provided by the UCLA AIDS Institute Virology Core Facility as institutional review board exempt materials without any demographic information.

### Chimeric Antigen Receptor Gene and Lentiviral Vector Construction

The lentiviral vector pCCLcMNDU3c-X2, gift of D. B. Kohn, was first modified to contain the sequence for the fixed regions (except the leader and single chain antibody sequences) of a previously reported human CAR, consisting of an IgG₄-based spacer (additionally containing a silent mutation creating a unique Apa I restriction site), CD8 transmembrane domain, co-signaling domain from 4-1BB (CD137), and signaling domain from the human CD3 complex ζ chain (CD247). Additionally, sequences for the P2A ribosomal skip sequence with a furin cleavage site followed by the heat stable antigen murine CD24 reporter (HSA) were inserted immediately downstream of the CAR sequences. This modified vector was digested with Hpa I and Apa I restriction enzymes, and PCR amplified products of the leader-single chain antibody sequences were inserted using the In-Fusion Cloning Kit (Takara, Mountain View, CA), followed by sequence confirmation of the final vectors. Lentiviral stocks were produced by co-transfection of HEK-293T cells with these constructs with Vesicular Stomatitis Virus envelope glycoprotein G protein and HIV-based packaging vectors, and stored in aliquots at −80° C. until use.

### CAR Transduction of Primary C8TLs

Cells were transduced with the CAR delivery lentiviral vectors . Briefly,

polystyrene 24-well tissue culture plates were pre-coated with RetroNectin (Takara Bio, Mountain View, CA). Lentiviral vector at a concentration of ˜50 ng p24 antigen in 100 μL volume was added and centrifuged at 2000 g for 2 hours. 10⁶ cells were then added for transduction, and maintained in RPMI 1640 supplemented with L-glutamine (2 mM), penicillin (100 U/mL), streptomycin (100 U/mL), heat-inactivated fetal bovine serum (10%), HEPES buffer (10 mM), and 50 U/mL recombinant human interleukin-2 (R10-50). These cells were maintained and enriched with periodic restimulations using a goat anti-human F(ab)2 antibody (catalog #109-006-006, Jackson ImmunoResearch Laboratories), to at least 70% purity before use in functional testing experiments.

### Western Blot for CAR Expression

Transduced C8TLs were assessed for CAR expression by Western blot. Briefly, two million transduced cells were lysed and subjected to protein electrophoresis, followed by transfer onto a 0.45 μm polyvinylidene difluoride (PVDF) membrane (Millipore, Billerica, MA). The membrane was probed by using a mouse anti-human CD247 monoclonal antibody (#551033; BD Pharmingen, San Jose, CA) and the SuperSignal West Pico detection kit (Pierce, Rockford, IL), which yielded bands for both endogenous CD247 as well as higher molecular weight CD247-containing CAR proteins.

### Flow Cytometric Analysis for Cell Surface CAR Expression

Detection of cell surface CAR expression on transduced cells was performed using methods in the art. In brief, cells were stained with fluorescein isothiocyanate (FITC)-conjugated goat anti-human F(ab)2 antibody (catalog #109-006-003, Jackson ImmunoResearch Laboratories, West Grove, PA) or isotype control antibody, fixed, and analyzed by flow cytometry (MACSQuantVYB, Miltenyi, Sunnyvale, CA) for analysis of cell surface CAR expression (FlowJo, Ashland, OR).

### CMV Strains and Production of Viral Stocks

GFP-labeled TB40/E and TR strains of human cytomegalovirus (CMV) that express a GFP reporter under the SV40 promoter were derived from BAC DNA, gift of T. E. Shenk and E. A. Murphy. CMV stocks were generated following viral propagation in ARPE-19 cells (American Type Tissue Culture Collection) by ultracentrifugation procedures in the art and titrated on ARPE-19 cells by immunostaining using the IE1-specific monoclonal antibody p63-27, gift of William Britt, and the Vectastain ABC kit (Vector Laboratories) according to the manufacturer's instructions.

### Flow Cytometric Analysis of Intracellular Cytokines and Cell Surface CD107a Expression by CAR-Transduced Primary C8TLs

ARPE-19 cells were seeded in 24-well plates at 2×10⁵ cells/well, and 24 hours later were infected with CMV using multiplicity of infection (MOI) of 3, with mock-infected cells as a negative control. Five days later, when the cells were uniformly infected as reflected by GFP expression, 10⁶ CAR-transduced primary C8TLs were added and incubated for an additional hour; stimulation with leukocyte activation cocktail (BD Biosciences, San Jose, CA) served as a positive control for activation. Brefeldin A and monensin (0.5 μl Golgi Plug and 0.5 μl Golgi Stop, BD Biosciences, San Jose, CA) and allophycocyanin (APC)-conjugated anti-CD107a antibody (catalog #328620, Biolegend, San Diego, CA) antibody were then added. After 5 hours of co-incubation, the CAR-transduced C8TLs were removed from each well for analysis. Surface staining was then performed with anti-human antibodies including anti-IgG F(ab)2 conjugated with fluorescein isothiocyanate (FITC) (catalog ##109-096-003, Jackson ImmunoResearch Laboratories), anti-CD8 conjugated with phycoerythrin (PE) (catalog #300908, Biolegend, San Diego, CA), followed by fixation and permeabilization (Cytofix/Cytoperm, BD Biosciences, San Jose, CA), followed by intracellular cytokine staining with anti-human antibodies including anti-interferon-γ conjugated with Alexa Fluor-647 (catalog #506507, Biolegend, San Diego, CA) and anti-tumor necrosis factor-α conjugated with Alexa Fluor-700 (catalog #502928, Biolegend, San Diego, CA). Cells were analyzed by FACSCelesta (BD Biosciences, San Jose, CA) using FlowJo software (BD Biosciences, San Jose, CA).

### Flow Cytometric Analysis of Cell Proliferation

ARPE-19 cells were used as stimulating cells to test for CMV-mediated proliferation of CAR-transduced C8TLs, using methods in the art with the following modifications. In a 48-well plate, 10⁵ ARPE-19 cells were seeded one day before infection with HCMV TB40/E or TR at a MOI of 1.5 and cultured for 3 days to obtain >90% infection as seen by GFP fluorescence (or mock-infected as negative controls). CAR-transduced C8TLs were labeled with CellTrace Violet according to the manufacturer's directions (Thermo Fisher Scientific, Grand Island, NY), and 5×10⁵ CAR-transduced cells with 3×10⁶ irradiated feeder PBMC were added to each well and cultured in R10-50 for seven days with a medium exchange at three days. Anti-CD3 antibody stimulation served as a positive control. For analysis, 5×10⁶ cells were harvested from each well and co-stained for CD8 and human F(ab), fixed with 1% paraformaldehyde, and analyzed by flow cytometry gated on the CD8″ population (MACSQuant VYB, Miltenyi Biotech, Sunnyvale, CA and FlowJo software, BD Biosciences, San Jose, CA).

### Chromium Release Assay of Cytolytic Activity Against CMV-Infected Cells by CAR-Transduced Primary C8TLs

Killing of CMV-infected target cells by CAR-transduced primary C8TLs was tested in ⁵¹Cr release assays using methods in the art, with the following modifications. ARPE-19 cells were plated at 10⁶ per T25 tissue culture flask and infected the next day with CMV TB40/E or TR at a multiplicity of infection of 1.5 (or mock-infected as a negative control), then cultured for four days to achieve >90% infection by GFP expression. The cells were detached by incubation in 5 mM EDTA in phosphate buffered saline for 15 minutes at 37° C., then labeled with ⁵¹Cr for use in chromium release assays.

### Assessment of Suppression of CMV Replication by CAR-Transduced Primary C8TLs.

Suppression of CMV replication was assessed. ARPE-19 cells were seeded into 96-well plates at 2×10⁴ cells/well, and infected following day with CMV TB40/E or TR with an MOI of 1. After four days, 2×10⁵ CAR-transduced (or control mock-transduced) primary C8TLs were added to each well and imaged after 8 hours of co-incubation using an Axio Observer Z1 inverted fluorescence microscope equipped with a linear motorized stage (Carl Zeiss, Pleasanton, CA).

### RESULTS

### Genetic Construction of Chimeric Antigen Receptors Based on Neutralizing Antibodies Against CMV

The sequences of previously identified neutralizing monoclonal antibodies targeting different CMV PC proteins were utilized as targeting regions for chimeric antigen receptor design (Table 1). These included antibodies targeting epitopes in the gH subunit (21E9, 2-80, 18F10, 62-11), conformational epitopes in the UL128/130/131A subunits (1B2, 12E2, 21F6), and a linear epitope in UL128 (13B5), all of which had previously been confirmed to bind CMV-infected cells (not shown). Genes for single chain versions of these antibodies were incorporated into the backbone that we previously described for HIV-1-specific CARs, consisting of the single chain antibody targeting region, a spacer based on the IgG₄ constant region, the CD8 transmembrane domain, and cytoplasmic signaling domains from 4-1BB and CD3ζ (FIG. 2). These novel CAR genes were inserted into a lentiviral vector for transduction of primary C8TLs.

### The Anti-CMV Antibody-Based CARs are Expressed by Transduced Primary C8TLs

Primary CD8⁺ cells from healthy donors were transduced and tested for expression of the novel CARs. Western blot analysis for expression of CD3ζ demonstrated detection of CAR expression as a larger CD3-expressing protein in addition to the native CD3ζ protein (FIG. 4). Cell surface expression of the CARs was further confirmed by staining for the immunoglobulin domain (not present on native T cells) on the surface of the transduced cells and detection by flow cytometry (FIG. 5B). Both modes of detection revealed that all eight novel CARs were expressed by primary C8TLs.

### CAR-Transduced Primary C8TLs are Specifically Triggered to Release Cytokines and Upregulate Cell Surface CD107a by CMV-Infected Target Cells

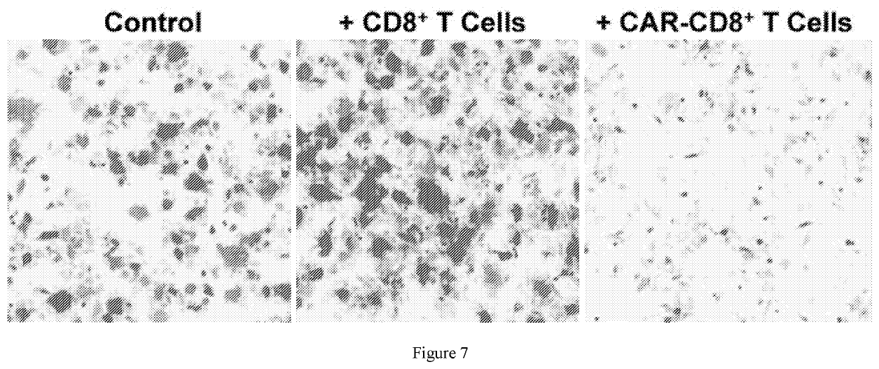

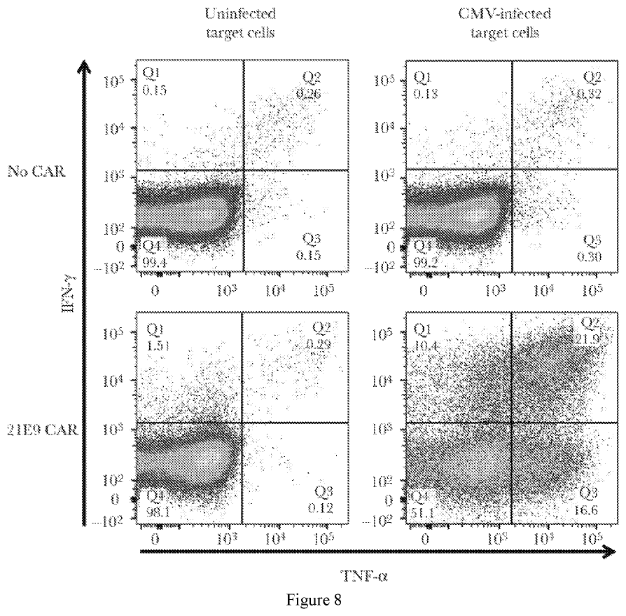

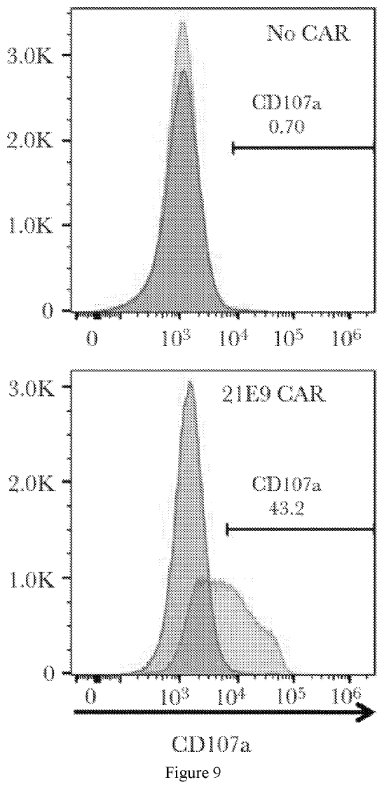

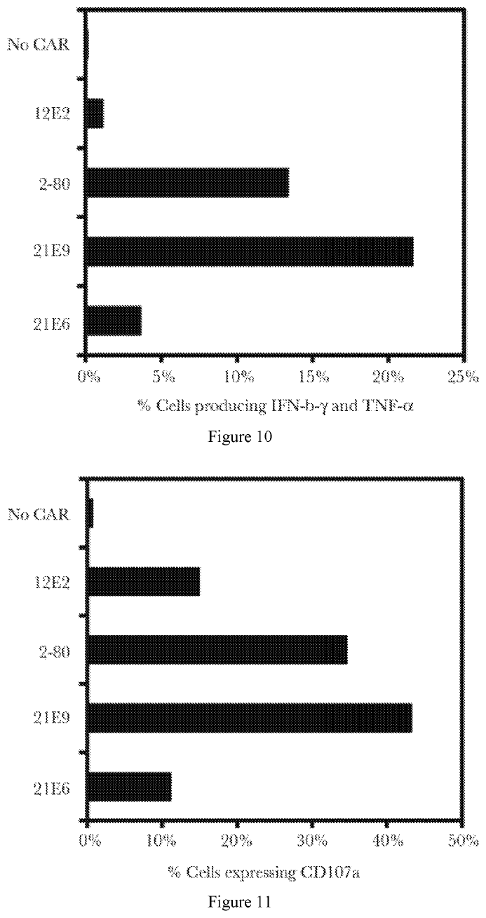

To test whether the CARs recognize their target proteins on the surface of CMV-infected cells, CAR-transduced primary C8TLs were exposed to acutely CMV TR-infected ARPE-19 cells and assessed for specific production of IFN-γ and TNF-α. Non-transduced C8TLs demonstrated minimal cytokine production in response to the ARPE-19 cells regardless of CMV infection. In contrast, at least two CARS, 21E9 and 2-80 (both targeting gH) showed specific cytokine release in response to CMV-infected but not CMV-uninfected cells (FIG. 8 and FIG. 10). Simultaneous evaluation for cell surface expression of the degranulation marker CD107a correlated to cytokine release (FIG. 9 and FIG. 11), again demonstrating that CARs 21E9 and 2-80, and to a lesser extent 12E2 and 21F6 (targeting the UL128/130/131A subunit), mediated CMV-specific cell triggering. Further studies focused on these four CARs. Similar results were seen with target ARPE-19 cells infected with CMV TB40/E infected cells (not shown).

### CAR-Transduced Primary C8TLs Proliferate in Response to CMV-Infected Target Cells

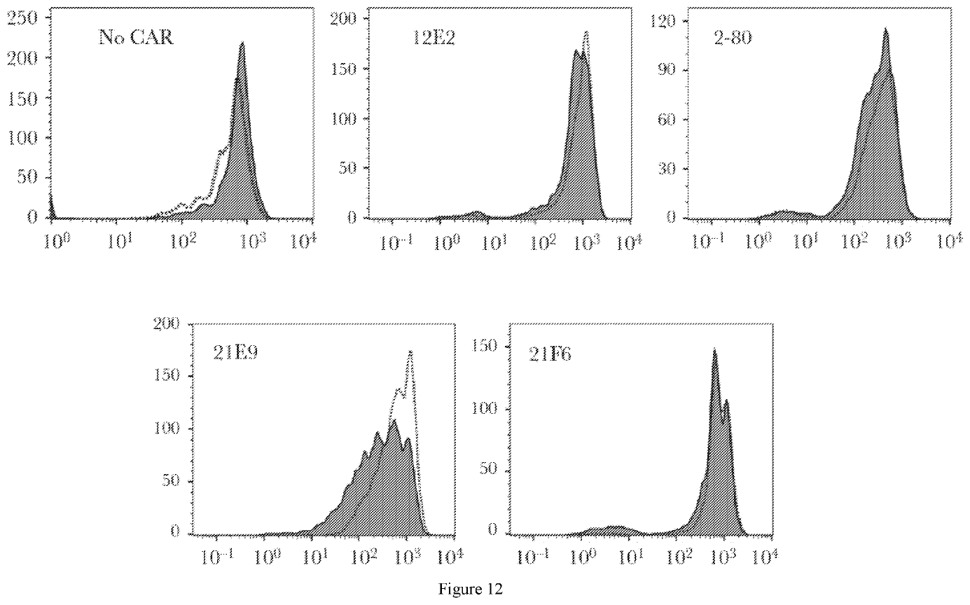

Further evaluating the function of CAR-transduced primary C8TLs, the proliferation of these cells in response to CMV-infected cells was tested (FIG. 12). The 21E9 CAR mediated modest proliferation when exposed to CMV-infected, but did not exhibit proliferation in response to uninfected cells. The other three CARs (2-80, 12E2, 21F6) mediated no appreciable proliferation. Thus at least one CAR conferred CMV-specific proliferation of transduced C8TLs.

CAR-Transduced primary C8TLs Mediate Modest Cytolysis of CMV-Infected Target Cells

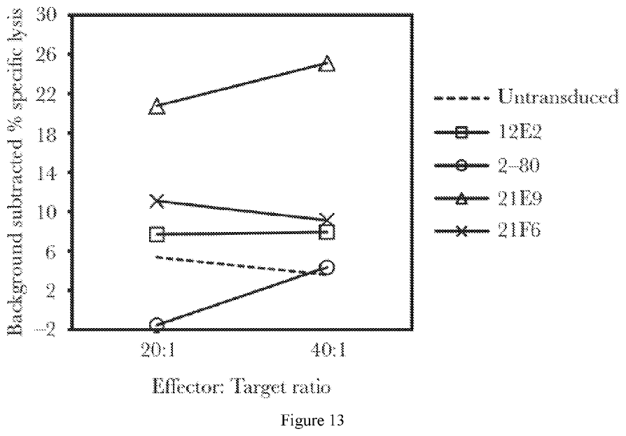

CMV-infected cells are intrinsically resistant to C8TL cytolysis. The capacity of CAR-redirected primary C8TLs to mediate CMV-specific cytolysis was tested by ⁵¹chromium release assays (FIG. 13). The only CAR that mediated consistent specific killing of CMV-infected target cells in multiple experiments was 21E9, which mediated cytolysis at modest levels using high effector:target ratios. The other CARs yielded lower and inconsistent levels of killing across multiple experiments.

### CAR-Transduced Primary C8TLs Suppress CMV Replication

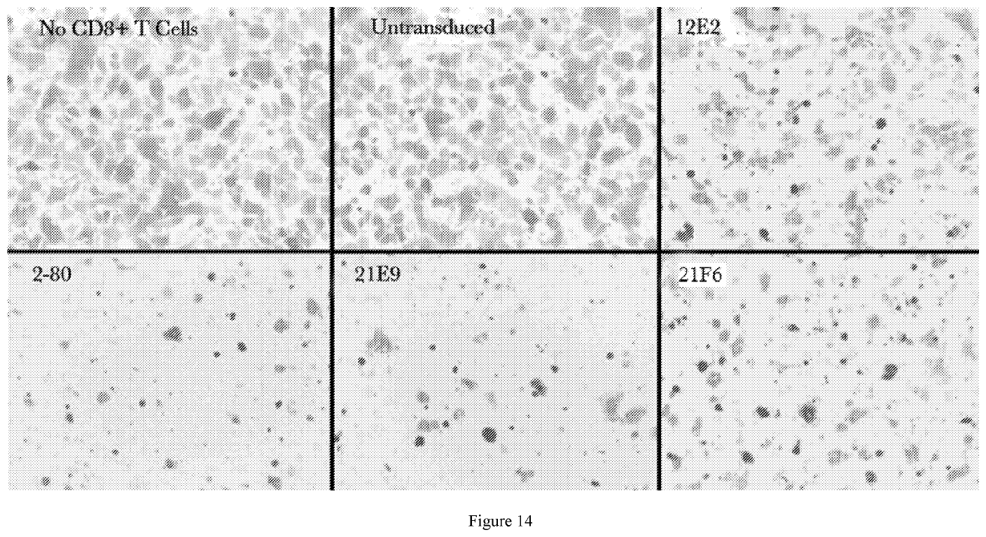

The antiviral activity of CAR-transduced C8TLs was evaluated by co-culture with acutely CMV-infected ARPE-19 cells using GFP-expressing CMV TR (FIG. 14). The four tested CARs all exhibited quantifiable antiviral activity with reduction of the concentration of fluorescent CMV-infected cells, even after a short incubation period of 8 hours. Across multiple experiments, CAR 21E9 mediated the most consistently potent antiviral activity. Similar results were seen with CMV TB40/E-infected ARPE-19 cells (not shown).

### DISCUSSION

To date, only one CMV-specific CAR has been reported in detail. That CAR targets CMV glycoprotein B (gB), and C8TL transfected with CAR-encoding RNA were shown to be CMV-specific by responding to infected cells by releasing IFN-γ and TNF-α and modestly upregulating surface CD107a. Although cells transfected with the prior art CAR killed target cells expressing recombinant gB, it was subsequently shown that they do not kill CMV-infected cells, presumably due to viral escape mechanisms against cytolysis.

Here, eight candidate CMV-specific CARs targeting other viral proteins on infected cells were designed and screened. These antibodies were previously generated in mice using a Modified Vaccinia Ankara Virus (MVA) vector expressing PC-sequences derived from TB40/E and found to bind the PC with high affinity. All the exemplified CMV-specific CARs exhibited functional activity. However, at least one of the CMV-specific CARs (based on the gH-specific antibody 21E9) was consistently active by multiple functional tests against cells infected with both CMV TB40/E and TR strains, which belong to two different gH genotypes.

While all the CMV-specific CARs exhibited adequate levels of expression on transduced cells, they varied in function. The 21E9-based CAR exhibited consistently superior activity in all functional tests, while the 2-80-based CAR showed activity in most assays, although it seemed to confer nonspecific background activity. The other CMV-specific CARs targeting other PC subunits demonstrated minimal activity overall. Whether this indicates that gH is a superior target for CARs than other PC proteins is unclear. 21E9 and 2-80 antibodies have about 10-fold less binding affinity than the other antibodies, but because the other two gH-specific antibodies 62-11 and 18F10 with similar affinity yielded poorly active CARs, affinity itself is probably not the major determinant of CAR activity. Target protein expression could be a factor; gH appears to be more abundant on CMV-infected cells than UL128, UL130, and UL131A. Another potential element is that the gH-specific antibodies utilized here recognize gH in multiple contexts including monomeric gH, gH bound to gL, and gH associated with the PC, perhaps allowing a broader target for binding and recognition. Finally, it is notable that 21E9 and 2-80 target a site on gH that is distinct from other gH-specific antibodies, suggesting that this epitope region may be more accessible to antibody in the context of a CMV-specific CAR.

The data herein shows modest but reproducible CAR-mediated C8TL killing of CMV-infected cells at levels approaching those observed by Rauser et al, who demonstrated cytolysis of infected cells by endogenous CMV-specific C8TLs. Overall, the role of infected cell killing for in vivo efficacy of CMV-specific CARs may not be critical, since adoptive transfer of native CMV-specific C8TLs has clearly demonstrated their antiviral effects, suggesting that targeted non-cytolytic mechanisms may be sufficient.

Beyond direct utility for anti-CMV therapy, a CMV-specific CAR may have other applications by harnessing the pathogenesis of chronic CMV infection. In normal immunocompetent hosts, this virus establishes a chronic lifelong infection that is mostly latent, but with frequent low level subclinical reactivations that stimulate relatively high levels of persistently circulating functional anti-CMV T cells. In this regard, CMV serves analogously to an endogenous vaccine that boosts and maintains cellular immunity against itself. A strategy being considered for cancer immunotherapy has been to harness this process by transducing CMV-specific T cells isolated from peripheral blood with an anti-CD19 CAR, thereby coupling the anti-tumor response to the anti-CMV response by creating bi-specific T cells recognizing both CMV and CD19. A functional anti-CMV CAR could achieve the same goal without the need to isolate CMV-specific T cells, via co-expression of the anti-CMV CAR with a T cell receptor or CAR targeting another virus or tumor.

Overall, the data herein indicate that CMV-specific CARs as disclosed herein, particularly the 21E9 CAR, are strong candidates for testing as immunotherapeutic intervention or prophylaxis for disseminated CMV infection and/or combination with T cell receptor or CAR gene immunotherapy for other diseases, given that the CMV-specific CARs exhibit CMV-targeted function in terms of triggering to release cytokines, proliferation, infected cell killing, and suppression of viral replication.

### REFERENCES

The following references are herein incorporated by reference in their entirety with the exception that, should the scope and meaning of a term conflict with a definition explicitly set forth herein, the definition explicitly set forth herein controls:
Staras S A, Dollard S C, Radford K W, Flanders W D, Pass R F, Cannon M J. Seroprevalence of cytomegalovirus infection in the United States, 1988-1994. Clin Infect Dis 2006; 43:1143-51.
Majeed A, Latif A, Kapoor V, et al. Resistant Cytomegalovirus Infection in Solid-organ Transplantation: Single-center Experience, Literature Review of Risk Factors, and Proposed Preventive Strategies. Transplantation proceedings 2018; 50:3756-62.
El Helou G, Razonable R R. Letermovir for the prevention of cytomegalovirus infection and disease in transplant recipients: an evidence-based review. Infection and drug resistance 2019; 12:1481-91.
Riddell S R, Walter B A, Gilbert M J, Greenberg P D. Selective reconstitution of CD8+ cytotoxic T lymphocyte responses in immunodeficient bone marrow transplant recipients by the adoptive transfer of T cell clones. Bone marrow transplantation 1994; 14 Suppl 4:S78-84.
Riddell S R, Watanabe K S, Goodrich J M, Li C R, Agha M E, Greenberg P D. Restoration of viral immunity in immunodeficient humans by the adoptive transfer of T cell clones. Science 1992; 257:238-41.
Walter E A, Greenberg P D, Gilbert M J, et al. Reconstitution of cellular immunity against cytomegalovirus in recipients of allogeneic bone marrow by transfer of T-cell clones from the donor. N Engl J Med 1995; 333: 1038-44.
Proff J, Walterskirchen C, Brey C, et al. Cytomegalovirus-Infected Cells Resist T Cell Mediated Killing in an HLA-Recognition Independent Manner. Frontiers in microbiology 2016; 7:844.
Full F, Lehner M, Thonn V, et al. T cells engineered with a cytomegalovirus-specific chimeric immunoreceptor. J Virol 2010; 84:4083-8.
Hahn G, Revello M G, Patrone M, et al. Human cytomegalovirus UL131-128 genes are indispensable for virus growth in endothelial cells and virus transfer to leukocytes. J Virol 2004; 78:10023-33.
Wang D, Shenk T. Human cytomegalovirus virion protein complex required for epithelial and endothelial cell tropism. Proc Natl Acad Sci USA 2005; 102:18153-8.
Chiuppesi F, Wussow F, Johnson E, et al. Vaccine-Derived Neutralizing Antibodies to the Human Cytomegalovirus gH/gL Pentamer Potently Block Primary Cytotrophoblast Infection. J Virol 2015; 89:11884-98.
Diamond D J, Chiuppesi F, Wussow F. MVA-gh/gL-PC vaccine derived antibodies neutralizing human cytomegalovirus infectivity and methods thereof. United States: City of Hope National Medical Center, 2018. U.S. Pat. No. 10,487,139.
Bennett M S, Joseph A, Ng H L, Goldstein H, Yang O O. Fine-tuning of T-cell receptor avidity to increase HIV epitope variant recognition by cytotoxic T lymphocytes. Aids 2010; 24:2619-28.
Ali A, Kitchen S G, Chen I S, Ng H L, Zack J A, Yang O O. HIV-1-Specific Chimeric Antigen Receptors Based on Broadly Neutralizing Antibodies. J Virol 2016; 90:6999-7006.
Logan A C, Nightingale S J, Haas D L, Cho G J, Pepper K A, Kohn D B. Factors influencing the titer and infectivity of lentiviral vectors. Hum Gene Ther 2004; 15:976-88.
Yang S, Cohen C J, Peng P D, et al. Development of optimal bicistronic lentiviral vectors facilitates high-level TCR gene expression and robust tumor cell recognition. Gene Ther 2008; 15:1411-23.
Ali A, Jamieson B D, Yang O O. Half-genome human immunodeficiency virus type 1 constructs for rapid production of reporter viruses. J Virol Methods 2003; 110:137-42.
Murphy E, Yu D, Grimwood J, et al. Coding potential of laboratory and clinical strains of human cytomegalovirus. Proc Natl Acad Sci USA 2003; 100:14976-81.
O'Connor C M, Murphy E A. A myeloid progenitor cell line capable of supporting human cytomegalovirus latency and reactivation, resulting in infectious progeny. J Virol 2012; 86:9854-65.
Britt W J. Human cytomegalovirus: propagation, quantification, and storage. Current protocols in microbiology 2010; Chapter 14:Unit 14E 3.
Wussow F, Chiuppesi F, Martinez J, et al. Human cytomegalovirus vaccine based on the envelope gH/gL pentamer complex. PLOS Pathog 2014; 10:e1004524.
Andreoni M, Faircloth M, Vugler L, Britt W J. A rapid microneutralization assay for the measurement of neutralizing antibody reactive with human cytomegalovirus. J Virol Methods 1989; 23:157-67.
Yang O O, Kalams S A, Rosenzweig M, et al. Efficient lysis of human immunodeficiency virus type 1-infected cells by cytotoxic T lymphocytes. J Virol 1996; 70:5799-806.
Bennett M S, Ng H L, Dagarag M, Ali A, Yang O O. Epitope-dependent avidity thresholds for cytotoxic T-lymphocyte clearance of virus-infected cells. J Virol 2007; 81:4973-80.
Stripecke R, Gerasch L, Theobald S, et al. CAR T Cells Targeted with a High Affinity Scfv Against the HCMV Glycoprotein Gb As Adoptive T Cell Therapy after Hematopoietic Stem Cell Transplantation. Blood 2016; 128:5721.
Shedlock D J, Talbott K T, Wu S J, et al. Vaccination with synthetic constructs expressing cytomegalovirus immunogens is highly T cell immunogenic in mice. Human vaccines & immunotherapeutics 2012; 8:1668-81.
Buscher N, Paulus C, Nevels M, Tenzer S, Plachter B. The proteome of human cytomegalovirus virions and dense bodies is conserved across different strains. Medical microbiology and immunology 2015; 204:285-93.
Barrios Y, Knor S, Lantto J, Mach M, Ohlin M. Clonal repertoire diversification of a neutralizing cytomegalovirus glycoprotein B-specific antibody results in variants with diverse anti-viral properties. Mol Immunol 2007; 44:680-90.
Lantto J, Fletcher J M, Ohlin M. Binding characteristics determine the neutralizing potential of antibody fragments specific for antigenic domain 2 on glycoprotein B of human cytomegalovirus. Virology 2003; 305:201-9.
Lantto J, Lindroth Y, Ohlin M. Non-germ-line encoded residues are critical for effective antibody recognition of a poorly immunogenic neutralization epitope on glycoprotein B of human cytomegalovirus. Eur J Immunol 2002; 32:1659-69.
Tempest P R, White P, Buttle M, Carr F J, Harris W J. Identification of framework residues required to restore antigen binding during reshaping of a monoclonal antibody against the glycoprotein gB of human cytomegalovirus. International journal of biological macromolecules 1995; 17:37-42.
Ohlin M, Owman H, Rioux J D, Newkirk M M, Borrebaeck C A. Restricted variable region gene usage and possible rheumatoid factor relationship among human monoclonal antibodies specific for the AD-1 epitope on cytomegalovirus glycoprotein B. Mol Immunol 1994; 31:983-91.
Newkirk M M, Gram H, Heinrich G F, Ostberg L, Capra J D, Wasserman R L. Complete protein sequences of the variable regions of the cloned heavy and light chains of a human anti-cytomegalovirus antibody reveal a striking similarity to human monoclonal rheumatoid factors of the Wa idiotypic family. J Clin Invest 1988; 81:1511-8.
Gilbert M J, Riddell S R, Plachter B, Greenberg P D. Cytomegalovirus selectively blocks antigen processing and presentation of its immediate-early gene product. Nature 1996; 383:720-2.
Rauser G, Einsele H, Sinzger C, et al. Rapid generation of combined CMV-specific CD4+ and CD8+ T-cell lines for adoptive transfer into recipients of allogeneic stem cell transplants. Blood 2004; 103:3565-72.
Sylwester A W, Mitchell B L, Edgar J B, et al. Broadly targeted human cytomegalovirus-specific CD4+ and CD8+ T cells dominate the memory compartments of exposed subjects. J Exp Med 2005; 202:673-85.
Wang X, Wong C W, Urak R, et al. CMVpp65 Vaccine Enhances the Antitumor Efficacy of Adoptively Transferred CD19-Redirected CMV-Specific T Cells. Clinical cancer research: an official journal of the American Association for Cancer Research 2015; 21:2993-3002.

All scientific and technical terms used in this application have meanings commonly used in the art unless otherwise specified.

Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises or consists of A” is used as a tool to avoid excess page and translation fees and means that in some embodiments the given thing at issue: comprises A or consists of A. For example, the sentence “In some embodiments, the composition comprises or consists of A” is to be interpreted as if written as the following two separate sentences: “In some embodiments, the composition comprises A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1-4. (canceled)

5. A cytomegalovirus specific chimeric antigen receptor (CMV-specific CAR), which comprises a single chain antibody sequence or fragment thereof having SEQ ID NO: 21, SEQ ID NO: 22, SEQ ID NO: 23, SEQ ID NO: 24, SEQ ID NO: 25, and SEQ ID NO: 26 as follows:
Formula H1
(SEQ ID NO: 21)
G-X1-X2-X3-X4-X5-X6-X7-X8-X9
wherein
X1 is F or Y,
X2 is S or T,
X3 is L or F,
X4 is S or T,
X5 is D, I, N, S, or T,
X6 is F, Y, or S,
X7 is G, Y, or W,
X8 is present or absent and if present X8 is L or I, and
X9 is present or absent and if present X9 is G;
Formula H2
(SEQ ID NO: 22)
I-X10-X11-X12-X13-X14-X15-X16
wherein
X10 is D, N, S, or W,
X11 is D, N, P, T, or W,
X12 is D, G, N, Y, or S,
X13 is D, G, or T,
X14 is D, G, N, or S,
X15 is E, K, S, or Y, and
X16 is present or absent and if present X16 is P or T;
Formula H3
(SEQ ID NO: 23)
X17-X18-X19-X20-X21-X22-X23-X24-X25-X26-X27-X28-
X29-X30-X31
wherein
X17 is A, S, or V,
X18 is R, N, or S,
X19 is E, G, K, P, R, or S,
X20 is G, H, K, L, Y, or W,
X21 is D, L, R, S, or Y,
X22 is D, F, G, L, S, or Y,
X23 is A, D, F, G, L, P, or Y,
X24 is A, D, I, Q, S, V, or Y,
X25 is present or absent and if present X25 is E, F, N, S, or Y,
X26 is present or absent and if present X26 is A, G, M, L, or P is,
X27 present or absent and if present X27 is D, L, or Y,
X28 is present or absent and if present X28 is A, F, L, or Y,
X29 is present or absent and if present X29 is D, F, G, or M,
X30 is present or absent and if present X30 is C, D, or Y, and
X31 is present or absent and if present X31 is S or Y;
Formula L1
(SEQ ID NO: 24)
X32-X33-X34-X35-X36-X37-X38-X39-X40-X41-X42
wherein
X32 is E, K, or Q,
X33 is G or S
X34 is I, L, or V,
X35 is D, G, S, or V,
X36 is D, H, N, S, or T,
X37 is D, N, S, or Y,
X38 is present or absent and if present X38 is D, G, or N,
X39 is present or absent and if present X39 is G, N, or Y,
X40 is present or absent and if present X40 is K, N, or S,
X41 is present or absent and if present X41 is F, Y, or T, and
X42 is present or absent and if present X42 is Y;
Formula L2
(SEQ ID NO: 25)
X43-X44-S
wherein
X43 is D, L, R, T, or Y, and
X44 is A, T, or V;
Formula L3
(SEQ ID NO: 26)
X45-X46-X47-X48-X49-X50-P-X51-T
wherein
X45 is S, Q, or W,
X46 is H, N, or Q,
X47 is D, G, S, or Y,
X48 is H, N, R, S, T, or Y,
X49 is E, H, K, R, S, or T,
X50 is D, F, L, S, V, or W, and
X51 is L, P, W, or Y.

6. The CMV-specific CAR according to claim 5, wherein:
a) SEQ ID NO: 21 is
(SEQ ID NO: 27)
GFSLSTYGIG,
(SEQ ID NO: 28)
GFSLTTSGLG,
(SEQ ID NO: 29)
GFTFSDYY,
(SEQ ID NO: 30)
GYTFTIYG,
(SEQ ID NO: 31)
GYTFTNFG,
(SEQ ID NO: 32)
GYTFTSYG,
(SEQ ID NO: 33)
GYTFTSYW,
(SEQ ID NO: 34)
GYTFTIYW,
or
(SEQ ID NO: 35)
GYTFTSYW;
b) SEQ ID NO: 22 is
(SEQ ID NO: 36)
IDPSDSET,
(SEQ ID NO: 37)
IDPSDSET,
(SEQ ID NO: 38)
IDPSDSET,
(SEQ ID NO: 39)
INTYTGEP,
(SEQ ID NO: 40)
ISDDGNYT,
(SEQ ID NO: 41)
ISNGGGST,
(SEQ ID NO: 42)
IWWDDDK,
or
(SEQ ID NO: 43)
IWWNDNK;
c) SEQ ID NO: 23 is
(SEQ ID NO: 44)
AREHYYGINPLLGC,
(SEQ ID NO: 45)
ARGWLLPVFAY,
(SEQ ID NO: 46)
ARKGYYGSSGYFDY,
(SEQ ID NO: 47)
ARRGDGLYSMDY
(SEQ ID NO: 48)
ARTGYFDV,
(SEQ ID NO: 49)
SNGYSSFAY,
(SEQ ID NO: 50)
VRPKRDFQYLYAMDY,
(SEQ ID NO: 51)
VRSLYDYDEGYYFDS,
or
(SEQ ID NO: 52)
ASSGTGAY;
d) SEQ ID NO: 24 is
(SEQ ID NO: 53)
ESIDSYGNSF,
(SEQ ID NO: 54)
KSVSTSGYSY,
(SEQ ID NO: 55)
QGISNY,
(SEQ ID NO: 56)
QSIGNN,
(SEQ ID NO: 57)
QSISDY,
(SEQ ID NO: 58)
QSLVHSNGNTY,
(SEQ ID NO: 59)
QSVSND,
(SEQ ID NO: 60)
QSISNN,
or
(SEQ ID NO: 61)
QSLLDSDGKTY;
e) SEQ ID NO: 25 is
(SEQ ID NO: 62)
DTS,
(SEQ ID NO: 63)
LAS,
(SEQ ID NO: 64)
RAS,
(SEQ ID NO: 65)
TVS,
(SEQ ID NO: 66)
YAS,
(SEQ ID NO: 67)
YTS,
or
(SEQ ID NO: 68)
LVS;
and/or
f) SEQ ID NO: 26 is
(SEQ ID NO: 69)
QHSRELPWT,
(SEQ ID NO: 70)
QNGHTFPPT,
(SEQ ID NO: 71)
QQDYSSPWT,
(SEQ ID NO: 72)
QQSNEDPLT,
(SEQ ID NO: 73)
QQSNRWPWT,
(SEQ ID NO: 74)
QQYSKLPYT,
(SEQ ID NO: 75)
SQSTHVPYT,
(SEQ ID NO: 76)
QQSNSWPLT,
or
(SEQ ID NO: 77)
WQGTHFPYT.

7. The CMV-specific CAR according to claim 6, wherein SEQ ID NO: 22 is INTYTGEP (SEQ ID NO: 39) and SEQ ID NO: 25 is YAS (SEQ ID NO: 66).

8. A method of treating, reducing, or inhibiting an infection by a cytomegalovirus in a subject, which comprises administering to the subject (a) an expression vector that encodes the CMV-specific CAR according to claim 5, or (b) one or more cells that are transduced with the expression vector.

9. A method of treating, reducing, or inhibiting an infection by a cytomegalovirus in a subject, which comprises transplanting one or more cells that express one or more CMV-specific CARs according to claim 5 to the subject.

10. An expression vector comprising a nucleic acid sequence encoding a CMV-specific CAR according claim 5.

11. A host cell comprising one or more expression vectors according to claim 10.

12. The host cell according to claim 11, wherein the host cell is a CD8+ T lymphocyte, hematopoietic stem cell, or a hematopoietic progenitor cell.

13. A cell that is the progeny of the host cell of claim 11.

14. The cell according to claim 11, wherein the cell expresses one or more chimeric antigen receptors encoded by the one or more expression vectors.

15. A method of treating, reducing, or inhibiting an infection by a cytomegalovirus in a subject, which comprises transplanting one or more cells according to claim 11, in the subject.

16. The method according to claim 15, wherein the subject is human and/or the cytomegalovirus is a human cytomegalovirus.

17. The method according to claim 15, wherein the subject has an immunodeficiency.

18. A method of killing cells infected with a cytomegalovirus, which comprises contacting the infected cells with one or more cells (a) that express one or more CMV-specific CARs according to claim 5, or (b) comprise an expression vector that encodes the one or more CMV-specific CARs.

19. A method of reducing replication of a cytomegalovirus in a cell or a subject, which comprises contacting the cell with or administering to the subject one or more cells (a) that express one or more CMV-specific CARs according to claim 5, or (b) comprise an expression vector that encodes the one or more CMV-specific CARs.

20. A CMV-specific CAR according to claim 5, wherein the single chain antibody or fragment thereof comprises SEQ ID NO: 30, SEQ ID NO: 39, SEQ ID NO: 46, SEQ ID NO: 59, SEQ ID NO: 66, and SEQ ID NO: 71.

21. The CMV-specific CAR according to claim 20, wherein the single chain antibody or fragment thereof comprises or consists of SEQ ID NO: 11 and SEQ ID NO: 12.

22. The CMV-specific CAR according to claim 20, which comprises or consists of SEQ ID NO: 89.

23. A method of treating, reducing, or inhibiting an infection by a cytomegalovirus in a subject, which comprises administering to the subject (a) an expression vector that encodes the CMV-specific CAR according to claim 20, or (b) one or more cells that are transduced with the expression vector.

24. The method according to claim 23, wherein the subject is human and/or the cytomegalovirus is a human cytomegalovirus.


SEQUENCE LISTING (91 sequences)

SEQ ID NO: 1 - protein, 118 aa, Artificial Sequence
  1B2 VH
EVQLVESGGVLVKPGGSLKLSCAASGFTFSDYYMYWVRQTPEKRLEWVATISDDGNYTNYPDSVKGRFTISRDNAKNNLYLQMSSLKSEDTAMYYCARGWLLPVFAYWGQGTLVTVSA

SEQ ID NO: 2 - protein, 107 aa, Artificial Sequence
  1B2 VL
DIVLTQSPATLSVTPGDSVSLSCRASQSIGNNLHWYQQKSHESPRLLIKYTSQSISGIPSRFSGSGSGTDFTLNINSVETEDFGVYFCQQSNRWPWTFGGGTKLEIK

SEQ ID NO: 3 - protein, 121 aa, Artificial Sequence
  21F6/54E11 VH
QIQLVQSGPELKKPGETVKISCKASGYTFTSYGMNWVKQAPGKGLKWMGWINTYTGEPTYADDFKGRFAFSLETSASTAYLQINNLKNEDTATYFCAREHYYGINPLLGCWGQGTTLTVSS

SEQ ID NO: 4 - protein, 107 aa, Artificial Sequence
  21F6/54E11 VL
DIQMTQTTSSLSASLGDRVTISCSASQGISNYLNWYQQKPDGTVKLLIYDTSSLHSGVPSRFSGSGSGTDYSLTISNLEPEDIATYYCQQYSKLPYTFGGGTKLEIK

SEQ ID NO: 5 - protein, 122 aa, Artificial Sequence
  12E2 VH
EVKLVESGGGLVQPGGSLKLSCATSGFTFSDYYMFWVRQTPEKKLEWVAYISNGGGSTYYPDTVKGRFTISRDNDKNTLYLQMSRLKSDDTALYYCVRPKRDFQYLYAMDYWGQGTSVTVSS

SEQ ID NO: 6 - protein, 111 aa, Artificial Sequence
  12E2 VL
DIVLTQSPASLAVSLGQRATISCRASKSVSTSGYSYMHWYQQKPGQSPKLLIYLASNLESGVPARFSGSGSGTDFTLNIHPVEDEDAATYYCQHSRELPWTFGGGTKLEIK

SEQ ID NO: 7 - protein, 123 aa, Artificial Sequence
  13B5 VH
QVTLKESGPGILKPSQTLSLTCSFSGFSLTTSGLGVGWIRQPSGKGLEWLAHIWWDDDKYFNPSLRNQLTISKDTSRNQVFLEITSVTTADTATYYCVRSLYDYDEGYYFDSWGQGTTLTVSS

SEQ ID NO: 8 - protein, 107 aa, Artificial Sequence
  13B5 VL
EIVMIQSPATLSVNPGDRVSLSCRASQSISDYLHWYQQKSHESPRLLIKYASQSISGIPSRFSGSGSGSDFTLSINSVEPEDVGVYYCQNGHTFPPTFGGGTKLEIK

SEQ ID NO: 9 - protein, 116 aa, Artificial Sequence
  18F10 VH
QVTLKESGPGILQPSQTLSLTCSFSGFSLSTYGIGIGWIRQPSGKGLEWLAHIWWNDNKNYNTALKSRLTISKDPSNNQVFLKIASVDTADTATYFCARTGYFDVWGAGTTVTVSS

SEQ ID NO: 10 - protein, 112 aa, Artificial Sequence
  18F10 VL
DVVLTQTPLSLPVSLGDQVSISCSSSQSLVHSNGNTYIHWYLQKPGQSPKLLIYTVSNRFSGVPDRFSGSGSGTDFTLKISRVEAEDLGLYFCSQSTHVPYTFGGGTKLEIK

SEQ ID NO: 11 - protein, 121 aa, Artificial Sequence
  21E9 VH
QIQLVQSGPELKKPGETVKISCKASGYTFTIYGMNWVKQAPGKGLKWMGWINTYTGEPTYADDFRGRFAFSLETSASTAYLQINNLKNEDTATYFCARKGYYGSSGYFDYWGQGTTLTVSS

SEQ ID NO: 12 - protein, 107 aa, Artificial Sequence
  21E9 VL
SIVMTQTPKFLLVSAGDRVTITCKASQSVSNDVSWYQQKPGQSPKLLIYYASNRYTGVPDRFTGSGYGTDFTFTISTVQAEDLAVYFCQQDYSSPWTFGGGTKLEIK

SEQ ID NO: 13 - protein, 116 aa, Artificial Sequence
  62-11 VH
QVQLQQPGAELVRPGASVKLSCKASGYTFTSYWMNWVKQRPGQGLEWIGMIDPSDSETHYNQMFKDKATLTVDKSSSTAYMQLSSLTSEDSAVYYCSNGYSSFAYWGQGTLVTVSV

SEQ ID NO: 14 - protein, 107 aa, Artificial Sequence
  62-11 VL
  misc_feature (2)..(2): Xaa can be any naturally occurring amino acid
  misc_feature (2)..(2): Xaa is V or I
DXQMTQTTSSLSASLGDRVTISCSASQGISNYLNWYQQKPDGTVKLLIYDTSSLHSGVPSRFSGSGSGTDYSLTISNLEPEDIATYYCQQYSKLPYTFGGGTKLEIK

SEQ ID NO: 15 - protein, 119 aa, Artificial Sequence
  2-80 VH
QIQLVQSGPELKKPGETVKISCKASGYTFTNFGMNWVKQAPGKGLKWMGWINTYTGEPTYADDFKGRFAFSLETSASTASLQINNLKNEDTATYFCARRGDGLYSMDYWGQGTSVTVSS

SEQ ID NO: 16 - protein, 111 aa, Artificial Sequence
  2-80 VL
DIVLTQSPASLAVSLGQRATISCRASESIDSYGNSFMYWYQQKPGQPPKLLIYRASNLESGIPARFSGSGSRTDFTLTINPVEADDVATYYCQQSNEDPLTFGAGTKLELK

SEQ ID NO: 17 - protein, 115 aa, Artificial Sequence
  4A3 VH
QVQLQQPGPELVRPGASVKLSCKASGYTFTIYWMNWVKQRPGQGLEWIGMIDPSDSETHYNQMFKDKATLTVDKSSSTAYMQLSSLTSEDSAVYYCASSGTGAYWGQGTLLTVSA

SEQ ID NO: 18 - protein, 112 aa, Artificial Sequence
  4A3 VL
DVVMTQTPLTLSVTIGQPASISCKSSQSLLDSDGKTYLNWLLQRPGQSPKRLIYLVSKLDSGVPDRFTGSGSGTDFTLKISRLEAEDLGVYYCWQGTHFPYTFGGGTKLEIK

SEQ ID NO: 19 - protein, 116 aa, Artificial Sequence; identical to SEQ ID NO: 13
  62-100 VH

SEQ ID NO: 20 - protein, 107 aa, Artificial Sequence
  62-100 VL
DIVLTQSPATLSVTPGDSVSLSCRASQSISNNLHWYQQKSHESPRLLIKYASQSISGIPSRFSGSGSGTDFTLSINSVETEDFGKYVCQQSNSWPLTFGSGTKLEIK

SEQ ID NO: 21 - protein, 10 aa, Artificial Sequence
  Formula H1
  misc_feature (2)..(2): Xaa is F or Y
  misc_feature (3)..(3): Xaa is S or T
  misc_feature (4)..(4): Xaa is L or F
  misc_feature (5)..(5): Xaa is S or T
  misc_feature (6)..(6): Xaa is D, I, N, S, or T
  misc_feature (7)..(7): Xaa is F, Y, or S
  misc_feature (8)..(8): Xaa is G, Y, or W
  misc_feature (9)..(9): Xaa is present or absent and if present Xaa is L or I
  misc_feature (10)..(10): Xaa is present or absent and if present Xaa is G
GXXXXXXXXX

SEQ ID NO: 22 - protein, 8 aa, Artificial Sequence
  Formula H2
  misc_feature (2)..(2): Xaa is D, N, S, or W
  misc_feature (3)..(3): Xaa is D, N, P, T, or W
  misc_feature (4)..(4): Xaa is D, G, N, Y, or S
  misc_feature (5)..(5): Xaa is D, G, or T
  misc_feature (6)..(6): Xaa is D, G, N, or S
  misc_feature (7)..(7): Xaa is E, K, S, or Y
  misc_feature (8)..(8): Xaa is present or absent and if present Xaa is P or T
IXXXXXXX

SEQ ID NO: 23 - protein, 15 aa, Artificial Sequence
  Formula H3
  misc_feature (1)..(1): Xaa is A, S, or V
  misc_feature (2)..(2): Xaa is R, N, or S
  misc_feature (3)..(3): Xaa is E, G, K, P, R, or S
  misc_feature (4)..(4): Xaa is G, H, K, L, Y, or W
  misc_feature (5)..(5): Xaa is D, L, R, S, or Y
  misc_feature (6)..(6): Xaa is D, F, G, L, S, or Y
  misc_feature (7)..(7): Xaa is A, D, F, G, L, P, or Y
  misc_feature (8)..(8): Xaa is A, D, I, Q, S, V, or Y
  misc_feature (9)..(9): Xaa is present or absent and if present Xaa is E, F, N, S, or Y
  misc_feature (10)..(10): Xaa is present or absent and if present Xaa is A, G, M, L, or P
  misc_feature (11)..(11): Xaa is present or absent and if present Xaa is D, L, or Y
  misc_feature (12)..(12): Xaa is present or absent and if present Xaa is A, F, L, or Y
  misc_feature (13)..(13): Xaa is present or absent and if present Xaa is D, F, G, or M
  misc_feature (14)..(14): Xaa is present or absent and if present Xaa is C, D, or Y
  misc_feature (15)..(15): Xaa is present or absent and if present Xaa is S or Y
XXXXXXXXXXXXXXX

SEQ ID NO: 24 - protein, 11 aa, Artificial Sequence
  Formula L1
  misc_feature (1)..(1): Xaa is E, K, or Q
  misc_feature (2)..(2): Xaa is G or S
  misc_feature (3)..(3): Xaa is I, L, or V
  misc_feature (4)..(4): Xaa is D, G, S, or V
  misc_feature (5)..(5): Xaa is D, H, N, S, or T
  misc_feature (6)..(6): Xaa is D, N, S, or Y
  misc_feature (7)..(7): Xaa is present or absent and if present Xaa is D, G, or N
  misc_feature (8)..(8): Xaa is present or absent and if present Xaa is G, N, or Y
  misc_feature (9)..(9): Xaa is present or absent and if present Xaa is K, N, or S
  misc_feature (10)..(10): Xaa is present or absent and if present Xaa is, F, Y, or T
  misc_feature (11)..(11): Xaa is present or absent and if present Xaa is Y
XXXXXXXXXXX

SEQ ID NO: 25 - protein, 3 aa, Artificial Sequence
  Formula L2
  misc_feature (1)..(1): Xaa is D, L, R, T, or Y
  misc_feature (2)..(2): Xaa is A, T, or V
XXS

SEQ ID NO: 26 - protein, 9 aa, Artificial Sequence
  Formula L3
  misc_feature (1)..(1): Xaa is S, Q, or W
  misc_feature (2)..(2): Xaa is H, N, or Q
  misc_feature (3)..(3): Xaa is D, G, S, or Y
  misc_feature (4)..(4): Xaa is H, N, R, S, T, or Y
  misc_feature (5)..(5): Xaa is E, H, K, R, S, or T
  misc_feature (6)..(6): Xaa is D, F, L, S, V, or W
  misc_feature (8)..(8): Xaa is L, P, W, or Y
XXXXXXPXT

SEQ ID NO: 27 - protein, 10 aa, Artificial Sequence
  18F10 VH CDR1
GFSLSTYGIG

SEQ ID NO: 28 - protein, 10 aa, Artificial Sequence
  13B5 VH CDR1
GFSLTTSGLG

SEQ ID NO: 29 - protein, 8 aa, Artificial Sequence
  1B2 and 12E2 VH CDR1
GFTFSDYY

SEQ ID NO: 30 - protein, 8 aa, Artificial Sequence
  21E9 VH CDR1
GYTFTIYG

SEQ ID NO: 31 - protein, 8 aa, Artificial Sequence
  2-80 VH CDR1
GYTFTNFG

SEQ ID NO: 32 - protein, 8 aa, Artificial Sequence
  54E11 VH CDR1
GYTFTSYG

SEQ ID NO: 33 - protein, 8 aa, Artificial Sequence
  62-11 VH CDR1
GYTFTSYW

SEQ ID NO: 34 - protein, 8 aa, Artificial Sequence
  4A3 VH CDR1
GYTFTIYW

SEQ ID NO: 35 - protein, 8 aa, Artificial Sequence
  62-100 VH CDR1
GYTFTSYW

SEQ ID NO: 36 - protein, 8 aa, Artificial Sequence
  4A3 VH CDR2
IDPSDSET

SEQ ID NO: 37 - protein, 8 aa, Artificial Sequence
  62-11 VH CDR2
IDPSDSET

SEQ ID NO: 38 - protein, 8 aa, Artificial Sequence
  62-100 VH CDR2
IDPSDSET

SEQ ID NO: 39 - protein, 8 aa, Artificial Sequence
  21E9, 54E11, and 2-80 VH CDR2
INTYTGEP

SEQ ID NO: 40 - protein, 8 aa, Artificial Sequence
  1B2 VH CDR2
ISDDGNYT

SEQ ID NO: 41 - protein, 8 aa, Artificial Sequence
  12E2 VH CDR2
ISNGGGST

SEQ ID NO: 42 - protein, 7 aa, Artificial Sequence
  13B5 VH CDR2
IWWDDDK

SEQ ID NO: 43 - protein, 7 aa, Artificial Sequence
  18F10 VH CDR2
IWWNDNK

SEQ ID NO: 44 - protein, 14 aa, Artificial Sequence
  54E11 VH CDR3
AREHYYGINPLLGC

SEQ ID NO: 45 - protein, 11 aa, Artificial Sequence
  1B2 VH CDR3
ARGWLLPVFAY

SEQ ID NO: 46 - protein, 14 aa, Artificial Sequence
  21E9 VH CDR3
ARKGYYGSSGYFDY

SEQ ID NO: 47 - protein, 12 aa, Artificial Sequence
  2-80 VH CDR3
ARRGDGLYSMDY

SEQ ID NO: 48 - protein, 8 aa, Artificial Sequence
  18F10 VH CDR3
ARTGYFDV

SEQ ID NO: 49 - protein, 9 aa, Artificial Sequence
  62-11 and 62-100 VH CDR3
SNGYSSFAY

SEQ ID NO: 50 - protein, 15 aa, Artificial Sequence
  12E2 VH CDR3
VRPKRDFQYLYAMDY

SEQ ID NO: 51 - protein, 15 aa, Artificial Sequence
  13B5 VH CDR3
VRSLYDYDEGYYFDS

SEQ ID NO: 52 - protein, 8 aa, Artificial Sequence
  4A3 VH CDR3
ASSGTGAY

SEQ ID NO: 53 - protein, 10 aa, Artificial Sequence
  2-80 VL CDR1
ESIDSYGNSF

SEQ ID NO: 54 - protein, 10 aa, Artificial Sequence
  12E2 VL CDR1
KSVSTSGYSY

SEQ ID NO: 55 - protein, 6 aa, Artificial Sequence
  54E11 and 62-11 VL CDR1
QGISNY

SEQ ID NO: 56 - protein, 6 aa, Artificial Sequence
  1B2 VL CDR1
QSIGNN

SEQ ID NO: 57 - protein, 6 aa, Artificial Sequence
  13B5 VL CDR1
QSISDY

SEQ ID NO: 58 - protein, 11 aa, Artificial Sequence
  18F10 VL CDR1
QSLVHSNGNTY

SEQ ID NO: 59 - protein, 6 aa, Artificial Sequence
  21E9 VL CDR1
QSVSND

SEQ ID NO: 60 - protein, 6 aa, Artificial Sequence
  62-100 VL CDR1
QSISNN

SEQ ID NO: 61 - protein, 11 aa, Artificial Sequence
  4A3 VL CDR1
QSLLDSDGKTY

SEQ ID NO: 62 - protein, 3 aa, Artificial Sequence
  54E11 and 62-11 VL CDR2
DTS

SEQ ID NO: 63 - protein, 3 aa, Artificial Sequence
  12E2 VL CDR2
LAS

SEQ ID NO: 64 - protein, 3 aa, Artificial Sequence
  2-80 VL CDR2
RAS

SEQ ID NO: 65 - protein, 3 aa, Artificial Sequence
  18F10 VL CDR2
TVS

SEQ ID NO: 66 - protein, 3 aa, Artificial Sequence
  21E9, 13B5, and 62-100 VL CDR2
YAS

SEQ ID NO: 67 - protein, 3 aa, Artificial Sequence
  1B2 VL CDR2
YTS

SEQ ID NO: 68 - protein, 3 aa, Artificial Sequence
  4A3 VL CDR2
LVS

SEQ ID NO: 69 - protein, 9 aa, Artificial Sequence
  12E2 VL CDR3
QHSRELPWT

SEQ ID NO: 70 - protein, 9 aa, Artificial Sequence
  13B5 VL CDR3
QNGHTFPPT

SEQ ID NO: 71 - protein, 9 aa, Artificial Sequence
  21E9 VL CDR3
QQDYSSPWT

SEQ ID NO: 72 - protein, 9 aa, Artificial Sequence
  2-80 VL CDR3
QQSNEDPLT

SEQ ID NO: 73 - protein, 9 aa, Artificial Sequence
  1B2 VL CDR3
QQSNRWPWT

SEQ ID NO: 74 - protein, 9 aa, Artificial Sequence
  54E11 and 62-11 VL CDR3
QQYSKLPYT

SEQ ID NO: 75 - protein, 9 aa, Artificial Sequence
  18F10 VL CDR3
SQSTHVPYT

SEQ ID NO: 76 - protein, 9 aa, Artificial Sequence
  62-100 VL CDR3
QQSNSWPLT

SEQ ID NO: 77 - protein, 9 aa, Artificial Sequence
  4A3 VL CDR3
WQGTHFPYT

SEQ ID NO: 78 - protein, 22 aa, Artificial Sequence
  Leader Sequence
MLLLVTSLLLCELPHPAFLLIP

SEQ ID NO: 79 - protein, 16 aa, Artificial Sequence
  Flexible Linker
SGGGGSGGGGSGGGGS

SEQ ID NO: 80 - protein, 229 aa, Artificial Sequence
  IgG4 hinge-CH2-CH3 region spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 25 aa, Artificial Sequence
  CD8 transmembrane region
DIYIWAPLAGTCGVLLLSLVITLYC

SEQ ID NO: 82 - protein, 42 aa, Artificial Sequence
  4-1BB co-signaling region
KRGRKKLLYIFKQPFMRPVQTTQEEDGCSCRFPEEEEGGCEL

SEQ ID NO: 83 - protein, 112 aa, Artificial Sequence
  CD3 zeta signaling region
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 671 aa, Artificial Sequence
  1B2_CAR
MLLLVTSLLLCELPHPAFLLIPEVQLVESGGVLVKPGGSLKLSCAASGFTFSDYYMYWVRQTPEKRLEWVATISDDGNYTNYPDSVKGRFTISRDNAKNNLYLQMSSLKSEDTAMYYCARGWLLPVFAYWGQGTLVTVSASGGGGSGGGGSGGGGSDIVLTQSPATLSVTPGDSVSLSCRASQSIGNNLHWYQQKSHESPRLLIKYTSQSISGIPSRFSGSGSGTDFTLNINSVETEDFGVYFCQQSNRWPWTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 676 aa, Artificial Sequence
  2-80_CAR
MLLLVTSLLLCELPHPAFLLIPQIQLVQSGPELKKPGETVKISCKASGYTFTNFGMNWVKQAPGKGLKWMGWINTYTGEPTYADDFKGRFAFSLETSASTASLQINNLKNEDTATYFCARRGDGLYSMDYWGQGTSVTVSSSGGGGSGGGGSGGGGSDIVLTQSPASLAVSLGQRATISCRASESIDSYGNSFMYWYQQKPGQPPKLLIYRASNLESGIPARFSGSGSRTDFTLTINPVEADDVATYYCQQSNEDPLTFGAGTKLELKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 679 aa, Artificial Sequence
  12E2_CAR
MLLLVTSLLLCELPHPAFLLIPEVKLVESGGGLVQPGGSLKLSCATSGFTFSDYYMFWVRQTPEKKLEWVAYISNGGGSTYYPDTVKGRFTISRDNDKNTLYLQMSRLKSDDTALYYCVRPKRDFQYLYAMDYWGQGTSVTVSSSGGGGSGGGGSGGGGSDIVLTQSPASLAVSLGQRATISCRASKSVSTSGYSYMHWYQQKPGQSPKLLIYLASNLESGVPARFSGSGSGTDFTLNIHPVEDEDAATYYCQHSRELPWTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 676 aa, Artificial Sequence
  13B5_CAR
MLLLVTSLLLCELPHPAFLLIPQVTLKESGPGILKPSQTLSLTCSFSGFSLTTSGLGVGWIRQPSGKGLEWLAHIWWDDDKYFNPSLRNQLTISKDTSRNQVFLEITSVTTADTATYYCVRSLYDYDEGYYFDSWGQGTTLTVSSSGGGGSGGGGSGGGGSEIVMIQSPATLSVNPGDRVSLSCRASQSISDYLHWYQQKSHESPRLLIKYASQSISGIPSRFSGSGSGSDFTLSINSVEPEDVGVYYCQNGHTFPPTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 674 aa, Artificial Sequence
  18F10_CAR
MLLLVTSLLLCELPHPAFLLIPQVTLKESGPGILQPSQTLSLTCSFSGFSLSTYGIGIGWIRQPSGKGLEWLAHIWWNDNKNYNTALKSRLTISKDPSNNQVFLKIASVDTADTATYFCARTGYFDVWGAGTTVTVSSSGGGGSGGGGSGGGGSDVVLTQTPLSLPVSLGDQVSISCSSSQSLVHSNGNTYIHWYLQKPGQSPKLLIYTVSNRFSGVPDRFSGSGSGTDFTLKISRVEAEDLGLYFCSQSTHVPYTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 674 aa, Artificial Sequence
  21E9_CAR
MLLLVTSLLLCELPHPAFLLIPQIQLVQSGPELKKPGETVKISCKASGYTFTIYGMNWVKQAPGKGLKWMGWINTYTGEPTYADDFRGRFAFSLETSASTAYLQINNLKNEDTATYFCARKGYYGSSGYFDYWGQGTTLTVSSSGGGGSGGGGSGGGGSSIVMTQTPKFLLVSAGDRVTITCKASQSVSNDVSWYQQKPGQSPKLLIYYASNRYTGVPDRFTGSGYGTDFTFTISTVQAEDLAVYFCQQDYSSPWTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 674 aa, Artificial Sequence
  21F6_54E11_CAR
MLLLVTSLLLCELPHPAFLLIPQIQLVQSGPELKKPGETVKISCKASGYTFTSYGMNWVKQAPGKGLKWMGWINTYTGEPTYADDFKGRFAFSLETSASTAYLQINNLKNEDTATYFCAREHYYGINPLLGCWGQGTTLTVSSSGGGGSGGGGSGGGGSDIQMTQTTSSLSASLGDRVTISCSASQGISNYLNWYQQKPDGTVKLLIYDTSSLHSGVPSRFSGSGSGTDYSLTISNLEPEDIATYYCQQYSKLPYTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 669 aa, Artificial Sequence
  62-11_CAR
MLLLVTSLLLCELPHPAFLLIPQVQLQQPGAELVRPGASVKLSCKASGYTFTSYWMNWVKQRPGQGLEWIGMIDPSDSETHYNQMFKDKATLTVDKSSSTAYMQLSSLTSEDSAVYYCSNGYSSFAYWGQGTLVTVSSSGGGGSGGGGSGGGGSDIQMTQTTSSLSASLGDRVTISCSASQGISNYLNWYQQKPDGTVKLLIYDTSSLHSGVPSRFSGSGSGTDYSLTISNLEPEDIATYYCQQYSKLPYTFGGGTKLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR